(19) United States
(12) Patent Application Publication
(10) Pub. No.: US 2025/0249044 A1
(43) Pub. Date: Aug. 7, 2025

(54) IFNy AND TNFa CO-STIMULATION OF MESENCHYMAL STROMAL CELLS DERIVED FROM MINOR SALIVARY (LABIAL) GLANDS FOR THERAPEUTIC USE

(71) Applicant: Wisconsin Alumni Research Foundation, Madison, WI (US)

(72) Inventors: Sara McCoy, Middleton, WI (US); Jacques Galipeau, Madison, WI (US)

(73) Assignee: Wisconsin Alumni Research Foundation, Madison, WI (US)

(21) Appl. No.: 19/042,945

(22) Filed: Jan. 31, 2025

Related U.S. Application Data
(60) Provisional application No. 63/548,659, filed on Feb. 1, 2024.

(51) Int. Cl. A61K 35/35; A61P 1/02; C12N 5/077; C12N 5/0775

(52) U.S. Cl. CPC A61K 35/35; A61P 1/02; C12N 5/0667; C12N 5/0669; C12N 2501/24; C12N 2501/25

(57) ABSTRACT

Methods of preparing mesenchymal stromal cells (MSCs) through co-stimulation with interferon gamma (IFNγ) and tumor necrosis factor alpha (TNFα), and methods for using the co-stimulated MSCs to treat conditions associated with exocrine glands. Co-treatment of MSCs with IFNγ and TNFα significantly enhances their trophic secretome, preserves their immunomodulatory capacity compared to untreated MSCs, and maximizes their therapeutic efficacy. The MSCs may be allogeneic to the recipient.

### CROSS-REFERENCE TO RELATED APPLICATIONS

Priority is hereby claimed to provisional application Ser. No. 63/548,659, filed Feb. 1, 2024, which is incorporated herein by reference.

### FEDERAL FUNDING STATEMENT

This invention was made with government support under TR002374 and DE031340 awarded by the National Institutes of Health. The government has certain rights to the invention.

### BACKGROUND

Mesenchymal stromal cells (MSCs) are a cell population that is responsible for tissue regeneration and immune system modulation. They can also be manufactured as a cellular product and are commonly derived from tissues such as bone marrow, umbilical cord, or adipose tissue, and propagated ex vivo using established, clinically applicable methods. See Lin et al. “Cell therapy for salivary gland regeneration,” J. Dent. Res. 2011, 90, 341-346. MSCs promote tissue healing, likely due to their ability to provide important tissue-promoting morphogens and limit cell death secondary to tissue inflammation. It is now thought that the healing effects of MSCs are more likely derived from the MSC secretome, which has broad immunomodulatory and trophic activity, than from differentiation of MSCs into target cells (Le Blanc et al. “Multipotent mesenchymal stromal cells and the innate immune system,” Nat. Rev. Immunol. 2012, 12, 383-396). For example, bone marrow MSCs given intravenously to treat irradiated rat colons, support local epithelial regeneration through Wnt4 (Sémont et al. “Mesenchymal stem cell therapy stimulates endogenous host progenitor cells to improve colonic epithelial regeneration,” PLOS One 2013, 8, e70170). In mice, the effect is through Wnt3a. (Gong et al. “Mesenchymal stem cells stimulate intestinal stem cells to repair radiation-induced intestinal injury,” Cell Death Dis. 2016, 7, e2387.) Akin to intestinal stem cells, the self-renewal ability of salivary gland stem cells is dependent on extrinsic niche signals, including Wnt, R-spondin, and GDNF. See, for example, Maimets et al. “Long-Term In Vitro Expansion of Salivary Gland Stem Cells Driven by Wnt Signals,” Stem Cell Reports 2016, 6, 150-162.

Mesenchymal stromal cells are also important immunomodulatory cells that promote favorable immune cell profiles. For example, MSCs promote tissue regeneration and act as immunomodulators. MSCs expand regulatory T cells and suppress cytotoxic T cells, processes mediated through key proteins including indoleamine 2,3-dioxygenase (IDO), Programmed Death-Ligand 1 (PD-L1), and Intercellular Adhesion Molecule 1 (ICAM-1). See Selleri et al. “Cord-blood-derived mesenchymal stromal cells downmodulate CD4+ T-cell activation by inducing IL-10-producing Th1 cells,” Stem Cells Dev 2013, 22, 1063-1075. The immunosuppressive properties of MSCs are primarily thought to be enhanced with IFNγ treatment. The importance of mesenchymal stromal cells has been studied in autoimmune diseases, such as Sjögren's disease (SjD) where human umbilical cord MSCS injected systemically into NOD mice improves the hallmark salivary gland inflammation of this disease. See Sun et al. “Mesenchymal stem cell transplantation alleviates Sjögren's syndrome symptoms by modulating Tim-3 expression,” Int. Immunopharmacol. 2022, 111, 109152.

Traditionally, bone marrow-derived MSCs (BM-MSCs), umbilical cord-derived (UC-MSC), and adipose-derived MSCs (A-MSC) have been used to treat xerostomia. BM-MSCs given intraglandularly improve salivary flow in mice. (Lin et al. “Cell therapy for salivary gland regeneration,” J. Dent. Res. 2011, 90, 341-346.) Studies in animal models of SjD have demonstrated significant improvements in unstimulated salivary flow or disease activity after BM-MSC injection, UC-MSC, and A-MSC therapy. See, respectively, Khalili et al. “Mesenchymal stromal cells improve salivary function and reduce lymphocytic infiltrates in mice with Sjogren's-like disease,” PLOS One 2012, 7, e38615; Ruan et al. “Effect of mesenchymal stem cells on Sjögren-like mice and the microRNA expression profiles of splenic CD4+ T cells,” Exp. Ther. Med. 2017, 13, 2828-2838; Shi et al. “Mesenchymal stem cell transplantation ameliorates Sjögren's syndrome via suppressing IL-12 production by dendritic cells,” Stem Cell. Res. Ther. 2018, 9, 308; and Liu et al. “Adipose-mesenchymal stromal cells suppress experimental Sjögren syndrome by IL-33-driven expansion of ST2(+) regulatory T cells,” iScience 2021, 24, 102446.

These works suggest that MSCs are capable of providing morphogens supportive of salivary gland regeneration. Though the studies have been in mice, a human study involving 404 subjects with a variety of autoimmune diseases (72 SjD patients) received systemicallogeneic bone marrow and umbilical cord MSCs, showing a favorable safety profile. (Liang et al. “Safety analysis in patients with autoimmune disease receiving allogeneic mesenchymal stem cells infusion: a long-term retrospective study,” Stem Cell. Res. Ther. 2018, 9, 312.) Other MSC sources including systemic administration of dental pulp and olfactory tissue have been studied for treatment of xerostomia/SjD. See Matsumura-Kawashima et al. “Secreted factors from dental pulp stem cells improve Sjögren's syndrome via regulatory T cell-mediated immunosuppression,” Stem Cell. Res. Ther. 2021, 12, 182 and Rui et al “Olfactory ecto-mesenchymal stem cell-derived exosomes ameliorate murine Sjögren's syndrome by modulating the function of myeloid-derived suppressor cells,” Cell. Mol. Immunol. 2021, 18, 440-451, respectively.

Very few people have studied salivary gland-derived mesenchymal stromal cell immunobiology of therapeutic efficacy MSG-MSCs mice with SjD-like disease. See Xu et al. “Effect of Bone Morphogenetic Protein 6 on Immunomodulatory Functions of Salivary Gland-Derived Mesenchymal Stem Cells in Sjögren's Syndrome,” Stem Cells Dev 2018, 27, 1540-1548. Salivary gland tissue is a better source of MSCs than adipose tissue or bone marrow in head and neck cancer and xerostomic patients because of the ease of procurement and that, as a source tissue, it aligns with the therapeutic target tissue. Bone marrow aspirates result in 4% of patients experiencing unbearable pain and 32% of patients experiencing severe pain. (Lidén et al. “Procedure-related pain among adult patients with hematologic malignancies,” Acta Anaesthesiol. Scand. 2009, 53, 354-363.) Although rare, bone marrow biopsies can result in major adverse events or death. (Bain, B. J. “Morbidity associated with bone marrow aspiration and trephine biopsy—a review of UK data for 2004,” Haematologica 2006, 91, 1293-1294.) Liposuction requires general anesthesia and is associated with major complications such as pneumothorax or death. (Platt et al. “Deaths associated with liposuction: case reports and review of the literature,” J. Forensic Sci. 2002, 47, 205-207.) In contrast, the minor salivary gland biopsy is well tolerated, requiring only local lidocaine prior to a <1 cm incision in the inner lower lip. Post-procedure care consists of the low-risk regimen of ice and NSAIDs. No major organ or life-threatening risks are associated with this minor procedure.

These limitations highlight the potential of salivary-gland derived MSCs. The low-risk isolation procedure combined with the processing of these MSCs makes labial salivary gland MSCs an optimal source of MSCs for therapeutic use.

MSCs likely do not persist indefinitely after they are administered, and their tissue healing effects may instead be due to their secretome. Thus, multiple MSC injections may be needed to effectively treat chronic diseases. One way to accomplish this is by expanding MSCs and cryopreserving them until clinically needed. However, cryopreservation results in cellular injury, less effective immunomodulation, or senescence. (Platt et al. “Deaths associated with liposuction: case reports and review of the literature,” J. Forensic Sci. 2002, 47, 205-207; Guan et al. “Inducible indoleamine 2,3-dioxygenase 1 and programmed death ligand 1 expression as the potency marker for mesenchymal stromal cells,” Cytotherapy 2018, 20, 639-649). Maximizing MSC efficacy and recovery after cryopreservation is essential to successful MSC therapies. To address this need, the present disclosure develops novel methods for improving the characteristics of salivary gland-derived MSCs for therapeutic administration.

### SUMMARY

Disclosed herein is a novel approach to improve efficacy of mesenchymal stromal cells (MSCs) for therapeutic purposes. Interferon gamma (IFNγ) is currently used to increase MSCs immunomodulatory and trophic potential as well as recovery from cryopreservation. Our new data supports the superiority of combined IFNγ and tumor necrosis factor alpha (TNFα) therapy to stimulate MSC trophic potential.

Specifically, disclosed and claimed herein is a method of treating xerostomia in a subject, the method comprising administering to the subject a therapeutically effective amount of a composition comprising mesenchymal stromal cells (MSCs), wherein the MSCs are treated with a combination of interferon gamma (IFNγ) and tumor necrosis factor alpha (TNFα) prior to administration.

The co-treatment of MSCs with IFNγ and TNFα promotes an increased secretome of the MSCs. In one embodiment, the MSCs have an increased secretion of R-spondin 3 after being treated with the combination of IFNγ and TNFα.

The MSCs may be cryopreserved after being treated with the combination of IFNγ and TNFα, and are cryo-recovered prior to administration. The IFNγ and TNFα combination synergistically enhances immunomodulatory capacity of the MSCs. The MSCs after being cryo-recovered have an increased expression of one or more immunomodulatory factors as compared to MSCs not being treated with the combination of IFNγ and TNFα.

The MSCs are isolated from a tissue prior to being treated by the combination of IFNγ and TNFα. Non-limiting examples of the tissue include salivary gland, bone marrow, umbilical cord, and an adipose tissue. Preferably, the tissue is salivary gland.

The MSCs may be allogeneic, autologous or syngeneic to the subject.

The IFNγ and TNFα used for treating the MSCs may be human IFNγ and TNFα. In some embodiments, the IFNγ and TNFα may be recombinant IFNγ and TNFα.

In some embodiments, the xerostomia is associated with Sjögren's syndrome or graft-versus-host disease. In some embodiments, the xerostomia is age-related xerostomia. In some embodiments, the xerostomia is radiation-induced or medication-induced xerostomia. The medication may comprise one or more of antidepressant, anticholinergic, antihypertensive, antihistamine, and chemotherapy drugs. The subject to be treated may be a mammal, including a human.

The objects and advantages of the disclosure will appear more fully from the following detailed description of the preferred embodiment of the disclosure made in conjunction with the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. MSCs were placed in differentiation medium. The top left and top right panel are the untreated controls stained with Oil Red O and Alizarin Red, respectively. The bottom left panel MSCs were cultured in adipocyte differentiation medium and stained with Oil Red O. The bottom right panel MSCs were cultured in osteogenic differentiation medium and stained with Alizarin Red.

FIGS. 2A-2I. Transcriptome analysis of MSC(SG), MSC(BM), and MSC(AD). FIG. 2A is a principal component analysis (PCA) plot showing the clustering of transcriptomes from different MSC sources. FIG. 2B is a Venn diagram showing the number of unique and shared genes among the different MSC sources. FIG. 2C is a heatmap showing the differential expression of genes across samples. FIGS. 2D-2I show the results of enrichment analyses of MSCs by source. The dot plots show enrichment of reactome pathways in each comparison listed.

FIGS. 3A-3D. Enrichment analyses of MSCs by source. MSC(SG) includes only those derived from healthy controls. n=3 healthy control MSC(SG); n=3 MSC(BM); n=3 MSC(AD). Dot plots show enrichment of reactome pathways in each comparison listed.

FIGS. 4A-4B. The trophic secretome differs by MSC source and cytokine stimulation conditions. Healthy controls of MSC(BM) (n=3), MSC(AD) (n=3), and MSC(SG) (n=9) were cultured with (i) vehicle; (ii) 10 ng/ml IFNγ; (iii) 10 ng/mL TNFα; (iv) 10 ng/ml IFNγ+10 ng/mL TNFα. Conditioned media were saved for ELISA. FIG. 4A. Secretome proteins from different sources of MSC were compared under vehicle treatment conditions; FIG. 4B. Secretome proteins from different sources of MSC were compared under IFNγ/TNFα treatment conditions.

FIGS. 5A-5C. The trophic secretome differs by MSC source and cytokine stimulation conditions. MSC(BM) (n=3), MSC(AD) (n=3), MSC(SG) (n=10) were cultured with (i) vehicle; (ii) 10 ng/mL IFNγ; (iii) 2.5 ng/ml TGFβ; (iv) 10 ng/ml TNFα; (v) 10 ng/ml IFNγ+2.5 ng/ml TGFβ; (vi) 10 ng/mL IFNγ+10 ng/mL TNFα. In addition to healthy controls, n=3 SjD and n=3 sicca controls were included in the analyses. Conditioned media were saved for ELISA. FIG. 5A. Secretome proteins from different sources of MSC were compared under vehicle treatment conditions; FIG. 5B. Secretome proteins from different sources of MSC were compared under IFNγ/TNFα treatment conditions; FIG. 5C. Secretome proteins from different MSC sources were compared under IFNγ/TGFβ treatment conditions. Ordinary ANOVA was used for equal SDs.

FIGS. 6A-6E. IFNγ and TNFα combination synergistically enhances MSC(SG) immunomodulatory capacity. FIG. 6A is a flow cytometry graph showing the stepwise gating analysis of the MSC(SG) after cryo-recovery and staining. FIG. 6B shows the expression level of ICAM, IDO, and PD-L1 in MSC(SG) pre-licensed with IFNγ and TNFα, compared to vehicle, IFNγ, or TNFα treated conditions. FIG. 6C is a flow cytometry graph showing the stepwise gating analysis of the MSC(SG) following cryo-recovery and co-culture with PBMCs. FIGS. 6D and 6E show T-cells proliferation in co-culture with MSC(SG) pre-licensed with IFNγ and TNFα, compared to vehicle or IFNγ treated conditions. FIG. 6E presents the results from FIG. 6D normalized to total number of MSCs surviving in the co-culture conditions.

FIGS. 7A-7B. The dose of TNFα does not affect immunomodulatory marker expression or doubling time. FIG. 7A. MSC[SG] (n=4) were cultured with (i) 60 ng/ml IFNγ and 10 ng/ml TNFα (IT10); (ii) 60 ng/mL IFNγ and 30 ng/mL TNFα (IT30); (iii) 60 ng/mL IFNγ and 60 ng/ml TNFα (IT60). Flow cytometry was performed for the shown markers as in FIG. 6A-6E. Median fluorescence intensity was reported; FIG. 7B. Doubling time of MSCs after cryo-rescue comparing cytokine treatment conditions n=4, each except TNFα and IT30 (n=3, each).

FIGS. 8A-8F. MSC(SG) pre-licensed with IFNγ and/or TNFα are superior to MSC(SG) without pre-licensing to preserve salivary gland function after radiation in mice. FIG. 8A shows the workflow of mouse injections following radiation. FIG. 8B shows salivary flow of mice injected with vehicle MSCs from different tissue sources (MSC(SG), MSC(BM), and MSC(AD). FIG. 8C shows salivary flow of mice injected with MSC(SG) pre-licensed with IFNγ alone or IFNγ and TNFα (dual cytokine). FIG. 8D shows salivary flow of mice injected with MSCs by tissue source pre-licensed with dual cytokines. FIGS. 8E and 8F shows persistence of the injected MSC(SG) (vehicle, MSC(SG) pre-licensed with IFNγ alone or dual cytokines) in SG tissue.

FIGS. 9A-9C. MSC(SG) pre-licensed with both IFNγ and TNFα preserve salivary gland structure compared to vehicle or IFNγ treatment alone. FIG. 9A shows histological structure of allogeneic and syngeneic salivary gland tissues treated with MSC(SG) pre-licensed with both IFNγ and TNFα, compared to vehicle or IFNγ treatment alone. FIG. 9B shows duct sizes of the allogeneic versus syngeneic salivary gland tissues with vehicle MSC(SG) or MSC(SG) pre-licensed with dual cytokines. FIG. 9C shows duct sizes of allogeneic salivary gland tissues treated with MSC(SG) pre-licensed with both IFNγ and TNFα, compared to vehicle or IFNγ treatment alone.

FIG. 10. Graphical summary of the methods and key results of the Example.

### DETAILED DESCRIPTION

### Abbreviations and Definitions

GDNF=Glial cell derived neutrophic factor; ICAM=Intercellular adhesion molecule 1; IDO=Indoleamine 2,3-dioxygenase; IFNγ=Interferon-gamma; LGR5=leucine rich repeat containing G protein-coupled receptor 5; MSCs=Mesenchymal stromal cells; MSC(AD)=Adipose-derived mesenchymal stromal cells; MSC(BM)=Bone marrow-derived mesenchymal stromal cells; MSC(SG)=Salivary gland-derived mesenchymal stromal cells; MSG=Minor salivary gland; PD-L1=Programmed death-ligand 1; pSS=Primary Sjögren's syndrome; RSPO=R-spodin; SjD=Sjögren's disease; TGFβ=Transforming growth factor beta; TNFα=Tumor necrosis factor alpha; TSG=TNF-stimulated gene; UC-MSCs=Umbilical cord-derived mesenchymal stromal cells.

The term “isolate” as used herein refers to removing matter (e.g., cells, proteins, etc.) from its natural milieu (i.e., that has been subject to human manipulation). As such, “isolated” does not reflect the extent to which the matter has been purified.

The terms “IFNγ” and “TNFα” as used herein are intended to include any IFNγ and TNFα proteins derived from a vertebrate source, including humans. Mature human IFNγ (UniProt Accession No. P01579) exists as a non-covalently linked homodimer of 20-25 kDa. Human IFNγ has about 90% amino acid (aa) sequence identity with rhesus IFNγ, 59%-64% with bovine, canine, equine, feline, and porcine IFNγ. Human TNFα (UniProt Accession No. P01375) consists of a 35 amino acid (aa) cytoplasmic domain, a 21 aa transmembrane segment, and a 177 aa extracellular domain (ECD). Within the ECD, human TNFα shares 97% aa sequence identity with rhesus TNFα, 71%-92% with bovine, canine, cotton rat, equine, feline, mouse, porcine, and rat TNFα. In certain embodiments, the terms “IFNγ” and “TNFα” includes any IFNγ and TNFα proteins that has substantially similar structure and function of naturally occurring IFNγ and TNFα proteins, or biologically active homologues or analogs of naturally occurring IFNγ and TNFα proteins. As such, the terms “IFNγ” and “TNFα” can include purified, partially purified, recombinant, mutated/modified and synthetic proteins.

Proteins and/or protein sequences are “homologous” when they are derived, naturally or artificially, from a common ancestral protein or protein sequence. A homologue of a given protein comprises, consists essentially of, or consists of, an amino acid sequence that is at least about 30%, 35%, 40%, 45%, 50%, 55%, 60%, 65%, 70%, 75%, 80%, 85%, 90%, 95%, or 99% identity to the given protein.

Analogs of a given protein refer to proteins that share a similar structure, function, or biological activity with the reference protein but may differ in amino acid sequence, origin, or molecular modifications. Analogs may include protein fragments, as well as proteins with one or more amino acid deletions, insertions, or substitutions. An analog of a given protein comprises, consists essentially of, or consists of an amino acid sequence with at least about 30%, 35%, 40%, 45%, 50%, 55%, 60%, 65%, 70%, 75%, 80%, 85%, 90%, 95%, or 99% sequence identity to the given protein.

Sequence “identity” refers to the number of exactly matching amino acids (expressed as a percentage) in a sequence alignment between two sequences of the alignment calculated using the number of identical positions divided by the greater of the shortest sequence or the number of equivalent positions excluding overhangs wherein internal gaps are counted as an equivalent position. For example, the polypeptides GGGGGG and GGGGT have a sequence identity of 4 out of 5 or 80%. For example, the polypeptides GGGPPP and GGGAPPP have a sequence identity of 6 out of 7 or 85%. In certain embodiments, any recitation of sequence identity expressed herein may be substituted for sequence similarity. Percent “similarity” is used to quantify the similarity between two sequences of the alignment. This method is identical to determining the identity except that certain amino acids do not have to be identical to have a match. Amino acids are classified as matches if they are among a group with similar properties according to the following amino acid groups: Aromatic—F Y W; hydrophobic—A VIL; Charged positive: R K H; Charged negative—D E; Polar—ST N Q.

Methods of obtaining IFNγ and TNFα are known in the art. For example, IFNγ and TNFα may be purified from cell sources using known methods. See e.g., U.S. Pat. No. 4,376,821; Aggarwal et al. “Human tumor necrosis factor. Production, purification, and characterization,” J Biol Chem. 1985, 25, 2345-2354. Alternatively, IFNγ and TNFα may be made by recombinant methods. The expression of IFNγ and TNFα using recombinant host cells, e.g., bacteria, yeast, mammalian cells has been widely reported. See e.g., Khalilzadeh et al. “Process development for production of recombinant human interferon-γ expressed in Escherichia coli,” J Ind Microbiol Biotechnol 2004, 31, 63-69; Volosnikova et al. “The Production of Soluble Human Gamma Interferon in the Escherichia coli Expression System with a Decrease in Cultivation Temperature,” Appl. Biochem. Microbiol. 2023, 59, 132-137; Damough et al. “Optimization of culture conditions for high-level expression of soluble and active tumor necrosis factor-α in E. coli,” Protein Expr Purif. 2021, 179, 105805; Seow et al. “Expression, biological activity and kinetics of production of recombinant ovine TNF-α,” Vet. Immunol. Immunopathol. 1995, 44, 279-291.

As used herein, “treating” MSCs with a combination of IFNγ and TNFα refers to exposing, contacting, or incubating MSCs with IFNγ and TNFα, either simultaneously or sequentially, preferably simultaneously, under conditions sufficient to modulate the biological properties or functions of the MSCs. Such modulation may include activating or enhancing specific biological responses in the MSCs, such as increasing their immunomodulatory activity, promoting the secretome, or upregulating specific gene expression. The treatment process may vary in terms of the concentration of IFNγ and TNFα, the duration of exposure, or the sequence of administration to achieve the desired MSC response.

As used herein, the terms “therapy,” “therapeutic,” “treating,” “treat,” or “treatment” of a disease, disorder, or condition broadly refer to managing a disease by arresting or reducing its progression or clinical symptoms, alleviating the condition, or causing regression of the disease or its symptoms. Therapy encompasses prophylaxis, prevention, treatment, cure, remedy, reduction, alleviation, and/or providing relief from a disease, signs, and/or symptoms of a disease. Therapy encompasses an alleviation of signs and/or symptoms in patients with ongoing disease signs and/or symptoms. Therapy also encompasses “prophylaxis” and “prevention.” Prophylaxis includes preventing disease occurring subsequent to treatment of a disease in a patient or reducing the incidence or severity of the disease in a patient. The term “reduced,” for purpose of therapy, refers broadly to the clinical significant reduction in signs and/or symptoms. Therapy includes treating relapses or recurrent signs and/or symptoms. Therapy encompasses but is not limited to precluding the appearance of signs and/or symptoms anytime as well as reducing existing signs and/or symptoms and eliminating existing signs and/or symptoms. Therapy includes treating chronic disease (“maintenance”) and acute disease. For example, treatment includes treating or preventing relapses or the recurrence of signs and/or symptoms.

The term “administering” as used herein refers to any mode of transferring, delivering, introducing, or transporting matter such as a pharmaceutical composition, to a subject. Modes of administration include, but are not limited to, oral administration, topical contact, intravenous, intraperitoneal, intramuscular, intranasal, and subcutaneous administration.

An “effective amount” or a “therapeutically effective amount” is an amount-either as a single dose or as part of a series of does-which at the dosage regimen applied yields the desired therapeutic effect, i.e., to reach a certain treatment goal. A therapeutically effective amount is generally an amount sufficient to provide a therapeutic benefit in the treatment or management of the relevant condition, or to delay or minimize one or more symptoms associated with the presence of the condition. The dosage will depend on various factors including patient and clinical factors (e.g., age, weight, gender, clinical history of the patient, severity of the disorder and/or response to the treatment), the nature of the disorder being treated, the particular composition to be administered, the route of administration, and other factors.

The term “subject” as used herein refers to a human or non-human animal, generally a mammal. Where the subject is a living human who is receiving medical care for a disease or condition, it is also addressed as a “patient.”

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 1 to 9, from 3.6 to 4.6, from 3.5 to 9.9, and so forth.

All references to singular characteristics or limitations of the present disclosure shall include the corresponding plural characteristic or limitation, and vice-versa, unless otherwise specified or clearly implied to the contrary by the context in which the reference is made.

All combinations of method or process steps as used herein can be performed in any order, unless otherwise specified or clearly implied to the contrary by the context in which the referenced combination is made.

The methods of the present disclosure can comprise, consist of, or consist essentially of the essential elements and limitations of the method described herein, as well as any additional or optional ingredients, components, or limitations described herein or otherwise useful in synthetic organic chemistry. The disclosure provided herein may be practiced in the absence of any element or step which is not specifically disclosed herein.

### The Method and Use

Disclosed herein is a method of preparing a composition comprising mesenchymal stromal cells (MSCs), the method comprising treating the MSCs with a combination of interferon gamma (IFNγ) and tumor necrosis factor alpha (TNFα). The method improves trophic properties of the MSCs by promoting secretome of the MSCs. In certain embodiments, secretion of R-spondin is increased after the stimulation. The MSCs composition may be cryopreserved after the stimulation, and the stimulation improves efficacy of the MSCs after the freezing and thawing process.

The MSCs may be isolated from a tissue including, but not limited to, minor salivary gland, bone marrow, umbilical cord, and an adipose tissue. Preferably, the MSCs are isolated from minor salivary gland. Collagenase AF-1 and NB-6 may be used for digesting the salivary gland biopsy sample to promote greater cell proliferation in the resulting cells. A neutral protease may be added with the collagenase AF-1, and a Hyaluronidase (e.g., Hylenex®) may be added with the collagenase NB-6.

Also disclosed herein are methods of using the MSCs co-stimulated with IFNγ and TNFα for therapeutic purposes. In certain embodiments, this disclosure relates to methods of treating a disease or condition comprising administering an effective amount of a composition comprising MSCs to a subject in need thereof, wherein the MSCs are prepared by the method disclosed herein. In certain embodiments, the MSCs are cryopreserved after pre-licensed with IFNγ and TNFα, and cryo-recovered for administration.

Non-limiting examples of the disease or condition that can be treated with the IFNγ and TNFα co-stimulated MSCs include exocrine glands associated diseases (e.g., Sjögren's syndrome and xerostomia), hematological diseases, graft-versus-host disease (GVHD), diabetes, inflammatory diseases, diseases in the liver, kidney, and lungs, cardiovascular, colon (e.g., Crohn's disease), bone (e.g., osteogenesis imperfecta), and cartilage, neurological, and autoimmune diseases.

In one version, the MSC composition is used to treat xerostomia.

In one embodiment, the xerostomia may be associated with Sjögren's syndrome or graft-versus-host disease (GVHD), which is likely caused by inflammation and a local cytokine milieu driving aquaporin mislocalization. The MSCs disclosed herein may mitigate the pathogenesis by reducing local epithelial cytokine production and suppressing T cell proliferation.

In another embodiment, the xerostomia may be age-related xerostomia, likely caused by a reduced proportion of acinar cells. The MSCs disclosed herein may mitigate the pathogenesis by supporting the regeneration of epithelial progenitor cells.

In another embodiment, the xerostomia is radiation-induced xerostomia.

In another embodiment, the xerostomia is medication-induced xerostomia. The medications may include antidepressant, anticholinergic, antihypertensive, antihistamine, and chemotherapy drugs. These medications may induce xerostomia by causing cholinergic, adrenergic, and histaminic blockade; a or β adrenergic antagonism; calcium dysregulation; tissue retention; and radiation-related tissue damage. The MSCs disclosed herein may mitigate the pathogenesis by producing prostaglandins that upregulate choline acetyltransferase and vesicular acetylcholine transporter, stimulating cholinergic pathways, and exerting trophic effects on epithelial progenitor cells.

The MSCs for treating the disease or condition may be allogeneic, autologous, or syngeneic to the subject. In certain embodiments, the MSCs are allogeneic to the subject. Thus, the treatment does not require obtaining tissue from the subject for treatment.

The MSC composition consists essentially of mesenchymal stem cells and saline. In certain embodiments, the MSCs may be incorporated into a polymer matrix, collagen, or gel (hydrogel) matrix prior to administration. In certain embodiments, the MSCs may be contained in a syringe.

### EXAMPLES

Local mesenchymal stromal cell (MSC) administration is a promising therapy for xerostomia. MSCs deploy their advantageous effects through their trophic secretome and immunomodulatory capabilities. These functions are enhanced with IFNγ pre-licensing, but the effects of TNFα pre-licensing are unknown. The objective of the Example was to compare MSCs by tissue source (MSC(BM), MSC(AD), and salivary gland-derived [MSC(SG)]) and by cytokine pre-licensing conditions. We used RNA sequencing and ELISA to determine key trophic and immunomodulatory features differing between human MSC(BM), MSC(AD), and MSC(SG). We used ELISA and flow cytometry of T-cell co-culture to define the effect of IFNγ and/or TNFα on MSC trophic secretome and immunomodulatory capacity. Finally, we studied salivary flow and glandular recovery with MSC injection in radiation-induced xerostomia mice.

Bulk RNA sequencing of MSC(BM), MSC(AD), and MSC(SG) revealed that they shared 85% of transcripts. Key differences included extracellular matrix production and response to cytokines in MSC(SG). Regardless of MSC source, dual stimulation of MSCs with IFNγ and TNFα produced an average of more than a 20-fold increase in R-Spondin 3 compared to vehicle conditions. Additionally, IFNγ and TNFα pre-licensing optimized immunomodulatory marker expression more than IFNγ alone. Intercellular adhesion molecule 1 (ICAM-1) increased 12-fold more, programmed death ligand 1 (PD-L1) increased 1.4-fold more, and indoleamine 2,3 dioxygenase (IDO) increased 2-fold more with IFNγ/TNFα pre-licensing than IFNγ alone. Both cytokine stimulation conditions resulted in a 1.2-fold decrease in T-cell proliferation. Gland structure and salivary flow are preserved in irradiated mice treated with MSC(SG) pre-licensed with IFNγ/TNFα. The Example demonstrates that MSC(SG) pre-licensed with both IFNγ and TNFα deploy advantageous functional cell attributes for salivary gland regenerative medicine.

### Introduction

Mesenchymal stromal cells (MSCs) reside in tissue stroma and impart potent immunomodulatory properties. MSCs expand regulatory T cells and suppress cytotoxic T cells, processes mediated through key proteins including indoleamine 2,3-dioxygenase (IDO), Programmed Death-Ligand 1 (PD-L1), and Intercellular Adhesion Molecule 1 (ICAM-1) (Angoulvant et al. “Human mesenchymal stem cells suppress induction of cytotoxic response to alloantigens,” Biorheology. 2004, 41, 469-476; Prevosto et al. “Generation of CD4+ or CD8+ regulatory T cells upon mesenchymal stem cell-lymphocyte interaction,” Haematologica. 2007, 92, 881-888; Selleri et al. “Cord-blood-derived mesenchymal stromal cells downmodulate CD4+ T-cell activation by inducing IL-10-producing Th1 cells,” Stem Cells Dev. 2013, 22, 1063-1075), among others. In addition to their immunosuppressive properties, MSCs also have trophic effects and support local progenitor niche cells. Marrow-derived MSCs (MSC(BM)) sustain LGR5+ epithelial stem cells in the gut via expression of Wnts, fibroblast growth factors (FGFs) and R-Spondins (Aoki et al. “Foxl1-expressing mesenchymal cells constitute the intestinal stem cell niche,” Cell Mol Gastroenterol Hepatol. 2016, 2, 175-188). MSC(BM) accelerate intestinal epithelial recovery following sub-lethal total-body radiation, likely through their secretome including Wnt2b and R-Spondin (François et al. “Adoptive transfer of mesenchymal stromal cells accelerates intestinal epithelium recovery of irradiated mice in an interleukin-6-dependent manner. Cytotherapy. 2012, 14, 1164-1170; Karpus et al. “Colonic CD90+ Crypt Fibroblasts Secrete Semaphorins to Support Epithelial Growth,” Cell Rep. 2019, 26, 3698-3708).

The immunomodulatory effects of MSCs are amplified with in vitro IFNγ pre-stimulation (or pre-licensing). (See François et al. “Human MSC suppression correlates with cytokine induction of indoleamine 2,3-dioxygenase and bystander M2 macrophage differentiation,” Mol Ther. 2012, 20, 187-195; Chinnadurai et al. “Immune dysfunctionality of replicative senescent mesenchymal stromal cells is corrected by IFNγ priming,” Blood Adv. 2017, 1, 628-643; Chinnadurai et al. “IDO-independent suppression of T cell effector function by IFN-gamma-licensed human mesenchymal stromal cells,” J Immunol. 2014, 192, 1491-1501.) Additionally, IFNγ treatment prior to cryopreservation improves post-thaw recovery and functional characteristics of MSCs, a critical advantage for therapeutic MSCs utilized more broadly. Recent studies established that dual stimulation with both IFNγ and TNFα synergistically enhances the immunomodulatory capacity of MSCs (Jin et al. “Interferon-γ and Tumor Necrosis Factor-α Polarize Bone Marrow Stromal Cells Uniformly to a Th1 Phenotype,” Sci Rep. 2016, 6, 26345; Montesinos et al. “Human Bone Marrow Mesenchymal Stem/Stromal Cells Exposed to an Inflammatory Environment Increase the Expression of ICAM-1 and Release Microvesicles Enriched in This Adhesive Molecule: Analysis of the Participation of TNF-α and IFN-γ,” J Immunol Res. 2020, 2020, U.S. Pat. No. 8,839,625; Zimmermann et al. “Pre-conditioning mesenchymal stromal cell spheroids for immunomodulatory paracrine factor secretion,” Cytotherapy. 2014, 16, 331-345; Lu et al. “Single-cell profiles of human bone marrow-derived mesenchymal stromal cells after IFN-γ and TNF-α licensing,” Gene. 2021, 771, 145347). Because radiation causes a local aberrant immune response comprising CD3+ T cells, granzyme B-stained cytotoxic T-cells, macrophages, and monocytes, immunomodulation might offset damage related to this robust inflammatory response (Teymoortash et al. “Lymphocyte subsets in irradiation-induced sialadenitis of the submandibular gland,” Histopathology. 2005, 47, 493-500). Other mechanisms implicated in radiation induced salivary gland damage include apoptosis and senescence or dysfunction of progenitor cells, indicating trophic factors might also mitigate radiation-induced damage (Jasmer et al. “Radiation-Induced Salivary Gland Dysfunction: Mechanisms, Therapeutics and Future Directions,” J Clin Med. 2020, 9). The effects of dual cytokine stimulation on the trophic secretome of MSCs remain unknown.

The unique and potent properties of MSCs, a therapy with low risk profile, make them a promising therapy to alleviate fibrotic and inflammatory diseases. Accordingly, MSCs have recently been deployed in studies as a treatment for radiation-induced xerostomia. These studies use adipose-derived MSCs (MSC(AD)) or IFNγ pre-licensed MSC(BM). (See Blitzer et al. “Marrow-Derived Autologous Stromal Cells for the Restoration of Salivary Hypofunction (MARSH): A pilot, first-in-human study of interferon gamma-stimulated marrow mesenchymal stromal cells for treatment of radiation-induced xerostomia,” Cytotherapy. 2023, 25, 1139-1144; Grønhøj et al. “Safety and Efficacy of Mesenchymal Stem Cells for Radiation-Induced Xerostomia: A Randomized, Placebo-Controlled Phase 1/2 Trial (MESRIX),” Int J Radiat Oncol Biol Phys. 2018, 101, 581-592.) The process of obtaining MSC(BM) and MSC(AD) can be associated with pain or other adverse effects. Bone marrow aspirates result in 4% of patients experiencing unbearable pain and 32% of patients experiencing severe pain (Degen et al. “Bone marrow examination: a prospective survey on factors associated with pain,” Ann Hematol. 2010, 89, 619-624; Lidén et al. “Procedure-related pain among adult patients with hematologic malignancies,” Acta Anaesthesiol Scand. 2009, 53, 354-363). Liposuction requires general anesthesia and is associated with major complications such as aspiration pneumonia, anesthesia reaction and cases of pneumothorax or death (Mentz et al. “Pneumothorax as a Complication of Liposuction.” Aesthet Surg J. 2020, 40, 753-758; Platt et al. “Deaths associated with liposuction: case reports and review of the literature,” J Forensic Sci. 2002, 47, 205-207). In contrast, the labial salivary gland (SG) biopsy, from which MSC(SG) s are collected, is well tolerated, requiring only local lidocaine prior to a <1 cm incision in the inner lower lip (Ike et al. “Bedside labial salivary gland biopsy (LSGBx: Lip biopsy): An update for rheumatologists.” Best Pract Res Clin Rheumatol. 2023, 37, 101839). Post-procedure care consists of the low-risk regimen of ice and NSAIDs. No major organ or life-threatening risks are associated with this minor and relatively low-cost procedure. Finally, SG might represent a better source of MSCs for xerostomia because its source tissue aligns with the therapeutic target tissue.

Our objective is to compare MSC(SG) to more traditional MSC sources including MSC(AD) and MSC(BM), evaluating the effects of cytokine stimulation (IFNγ and TNFα) on the trophic secretome and function of MSC(SG).

### Methods

### Samples

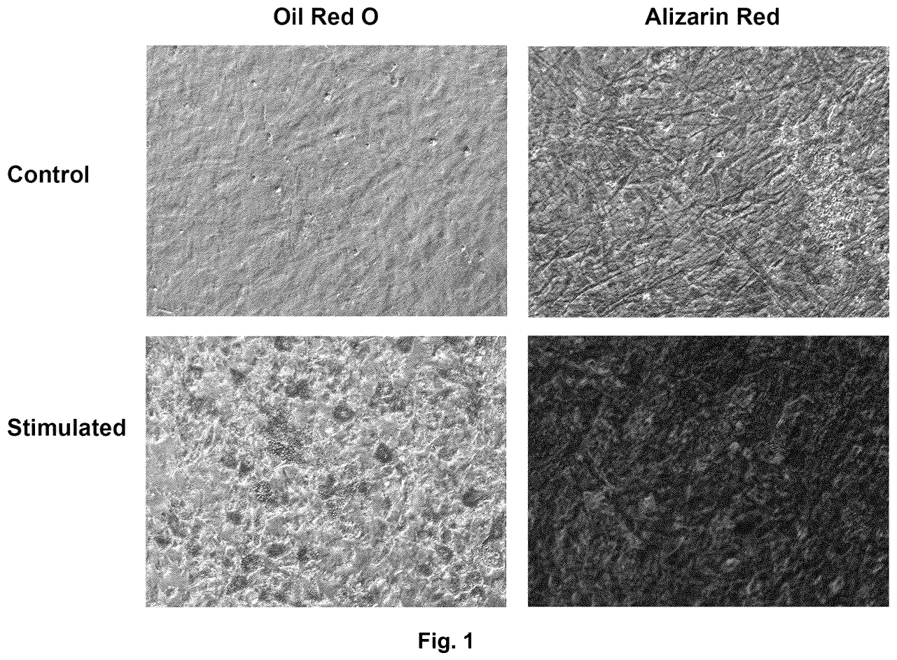

All the work described was performed under the University of Wisconsin Health Sciences IRB 2016-1545, 2018-0815 and 2022-1491 and are completed in accordance with the Declaration of Helsinki. Informed consent was obtained for all human subjects. MSC(AD) were obtained from intra-abdominal adipose tissue and MSC(BM) were sourced from bone marrow as previously described (Chinnadurai et al. “Potency Analysis of Mesenchymal Stromal Cells Using a Phospho-STAT Matrix Loop Analytical Approach,” Stem Cells. 2019, 37, 1119-1125) and were derived from deidentified healthy donors. MSC(SG) were obtained from labial salivary gland biopsies as previously described (Ike et al. “Bedside labial salivary gland biopsy (LSGBx: Lip biopsy): An update for rheumatologists,” Best Pract Res Clin Rheumatol. 2023, 37, 101839; McCoy et al. “Minor salivary gland mesenchymal stromal cells derived from patients with Sjögren's syndrome deploy intact immune plasticity,” Cytotherapy. 2021, 23, 301-310). MSCs met criteria as recommended by the International Society of Cell Therapy including adherence to plastic, differentiation capability (FIG. 1), and surface markers. Differentiation assays were performed using R&D Stem X Vivo adipogenic and osteogenic differentiation supplements, in accordance with manufacturer's protocols.

Demographics were not available for the deidentified MSC(BM) and MSC(AD) healthy donors. Demographics for MSC(SG) are shown in Table 1. Labial SG donors classified as healthy controls volunteered to donate labial salivary glands prior to radiation for head and neck cancer. Labial SGs were not involved in any active disease process. Sicca controls had dry eye and/or mouth symptoms so proceeded to labial SG biopsy, but they did not have any evidence of an autoimmune process driving their symptoms (based on labs and pathology). Sjögren's disease (SjD) subjects met 2016 ACR/EULAR criteria for disease.

TABLE 1
Demographics for MSC(SG).

| | HC | | Sicca | SjD | |
|---|---|---|---|---|---|
| | n = 4 | | n = 3 | n = 3 | |
| Age mean (SD) | 51 | (4) | 45(13) | 52 | (5) |
| Female n(%) | 1 | (33) | 3(100) | 3 | (100) |
| Race (European) n(%) | 3 | (100) | 3(100) | 2 | (66) |

SjD feature (n %)

| | | | | |
|---|---|---|---|---|
| Positive SSA antibody | — | — | 3 | (100) |
| Low complement | — | — | 1 | (33) |
| High RF | — | — | 2 | (66) |
| Labial SG focus score | — | — | 1.6 | (1.4) |

HC = healthy control; Sicca = dryness symptoms but no evidence of immune activity; SjD = Sjören's disease; SD = standard deviation.

### Cell Culture

Sixteen populations of cells (n=3 bone marrow (BM); n=3 omental adipose; n=3 sicca minor salivary gland (MSG), n=3 Sjögren's MSG and n=4 non-sicca MSG-derived mesenchymal stromal cells) were grown in base media with six different cytokine conditions. The base media was composed of Dulbecco's modified Eagle's medium (DMEM, cat. #10-017-CV, Corning, Corning, NY)+1% pen/strep (cat. #30-002-CI, Corning, Corning, NY)+1% L-glutamine (cat. #25-005-CI, Corning, Corning, NY)+10% human platelet lysate (hPL, cat. #PLTMax100R, Millcreek Life Sciences, Rochester, MN)+2 units/mL of heparin. Cells were seeded at such density as was necessary for their successful growth (˜3-5 K/cm² for BM, ˜7-10K for adipose and ˜1-4 K/cm² for MSG-derived MSCs) and grown for ˜2-8 days in 10-cm plates with 10 mL of media, with media changes performed every other day until the confluence was estimated to be ˜60-80%. At that point, the media was withdrawn, the cells were washed once with 7.5 mL of PBS and then 10 mL of serum-starving media was added (base media with 1% hPL, instead of 10%). After 24 hours, the serum-starving media was withdrawn and 10 mL of media in one of six cytokine conditions was added: (i) no cytokines (two plates); (ii) 10 ng/mL IFNγ (cat. #300-02, PeproTech, Cranbury, NJ); (iii) 2.5 ng/mL TGFβ (cat. #100-21, PeproTech, Cranbury, NJ); (iv) 10 ng/mL TNFα (cat. #570102, BioLegend, San Diego, CA); (v) 10 ng/ml IFNγ+2.5 ng/mL TGFβ; (vi) 10 ng/mL IFNγ+10 ng/mL TNFα—in each instance, with D10hPL media as the base. All plates were held for 48 hours, with bright-field images of a representative field documented at the very end of that time frame. All plates were cultured at 37° C. and 5% CO₂.

At time of harvest, all steps were performed on ice: the conditioned media (CM) was withdrawn and saved. Specifically, the conditioned media was spun at 4,000 rpm, for 10 min at 4° C. in order to remove any stray cells. After the spin, the CM was decanted into a fresh tube. Two aliquots, 1 mL each, were set up in microcentrifuge tubes. All the samples at −80° C. until further aliquoting and use in ELISA assays.

Keeping the plates on ice, each plate was washed with 7.5 mL PBS, and the PBS was aspirated; then the plate was tilted to almost 90° to the surface, to let residual buffer collect in one spot, and it was aspirated up. At that point, 600 μL of cold lysis buffer (from CST #9803 10× lysis buffer; but supplemented with 1 mM each of EDTA, vanadate, PMSF and sodium fluoride) was added; the plate was tilted to spread this; it was then scraped, going across the whole surface about 3-5 times, tilted to collect the fluid using the cell scraper in one little spot, and finally the lysate was collected into a cold labeled microcentrifuge tube on ice. These lysates were frozen at −80° C. Bright-field microscopy was used to ensure that the plates were devoid of cells after scraping is done. In the case of the ‘Veh RNA plate’, the only difference was that 1.25 mL of Zymo Research Tri Reagent (cat. #R2050, ZymoResearch, Irvine, CA) was added in place of 600 μL of lysis buffer.

### RNA-Seq

Sixteen populations of cells (n=3 MSC[BM]; n=3 omental MSC[AD]; n=3 sicca MSC[SG]; n=3 SjD MSC[SG]; and n=4 non-sicca MSC[SG]) were seeded at ˜3-5 K/cm² for BM, ˜7-10K for adipose and ˜1-4 K/cm² for SG-derived MSCs. After reaching 80% confluence, MSCs were serum starved for 24 hours. RNA isolation and purification was performed using the DirectZol RNA Miniprep Plus Kit (ZymoResearch, Irvine, CA) according to the manufacturer's instructions, with elution into 50 μL of provided ultrapure water. After assessment of RNA purity and concentration via absorbance at 200-300 nm by Nanodrop, the isolated RNA was provided to Novogene, Inc (Sacramento, CA) for sequencing using the Illumina platform. Briefly, Novogene isolated messenger RNA from total RNA with poly-T-oligo-attached magnetic beads. The library was constructed after fragmentation and cDNA synthesis and after end repair, A-tailing, size selection, adaptor ligation, amplification, and purification. A standard bioinformatic pipeline was deployed by data QC (error rate distribution, GD-content distribution, and data filtering), mapping to reference genome, gene expression quantification, and differential expression analyses. Novogene performed functional analysis including enrichment analysis.

### ELISA

To establish the trophic secretome, MSC(BM) (n=3), MSC(AD) (n=3), and MSC(SG) (n=9) were cultured as above until 80% confluence. MSCs were treated with IFNγ (10 ng/ml; PeproTech, Cranbury, NJ), TNFα (10 ng/ml; BioLegend, San Diego, CA), or TGFβ (2.5 ng/mL; PeproTech, Cranbury, NJ). After 48 hours of culture, the conditioned media was used for ELISA per the manufacturer's recommendations (GDNF [Innovative Research Novi MI], RSPO1 [CUSABIO, Houston, TX], RSPO3 [Innovative Research], Wnt1 [CUSABIO], Wnt2b [CUSABIO], Wnt3a [CUSABIO], Wnt4 [Raybiotech, Norcross, GA], Wnt5a [CUSABIO]) with dilutions shown in Table 2.

For ELISA studies, conditioned media was thawed, pipetted/diluted with manufacturer's recommended dilution buffer into PCR strip tubes before being dispensed from there in duplicate via multi-channel pipette into the columns of 96-well plates of ELISA kits, with wells pre-coated with capture antibodies for each analyte of interest. For example, a dilution factor of 2:3 refers to 150 μL of conditioned media plus 75 μL of diluent, and a 1:5 dilution factor refers to 45 μL of conditioned media plus 180 μL of diluent. Concentrations of the analytes in the original conditioned media were determined by using 4-parameter logistic regression in GraphPad Prism with interpolation based on the absorbance of the standards in the kits.

TABLE 2
Dilution factors of analytes for ELISA studies.

| | | Dilution factors | | | | | |
|---|---|---|---|---|---|---|---|
| | | | | | | IFNγ + | IFNγ + |
| Analyte | Vendor | Veh | IFNγ | TGFβ | TNFα | TGFβ | TNFα |
| GDNF | Innovative Research | 2:3 | 2:3 | 2:3 | 2:3 | 2:3 | 2:3 |
| | (Novi, MI) | | | | | | |
| RSPO1 | CUSABIO | 1:1 | 1:1 | 1:1 | 1:1 | 1:1 | 1:1 |
| | (Houston, TX) | | | | | | |
| RSPO3 | Innovative Research | 2:3 | 2:3 | 2:3 | 2:3 | 2:3 | 2:3 |
| | (Novi, MI) | | | | | | |
| Wnt1 | CUSABIO | 1:1 | 1:1 | 1:1 | 1:1 | 1:1 | 1:1 |
| | (Houston, TX) | | | | | | |
| Wnt2b | CUSABIO | 1:5 | 1:7 | 1:7 | 1:4 | 1:7 | 1:4 |
| | (Houston, TX) | | | | | | |
| Wnt3a | CUSABIO | 1:1 | 1:1 | 1:1 | 1:1 | 1:1 | 1:1 |
| | (Houston, TX) | | | | | | |
| Wnt4 | Raybiotech | 1:8 | 1:8 | 1:10 | 2:3 | 1:10 | 2:3 |
| | (Norcross, GA) | | | | | | |
| Wnt5a | CUSABIO | 1:1 | 1:1 | 1:1 | 1:1 | 1:1 | 1:1 |
| | (Houston, TX) | | | | | | |

### Flow Cytometry

Pre-licensed MSCs were cryo-recovered from liquid nitrogen storage by slowly resuspending the thawing cells in a-MEM media supplemented with 10% charcoal-stripped FBS, 1% Glutamax and 1% penicillin/streptomycin. The cells were cultured for 18 hours prior to harvest by trypisinization (0.05% trypsin/0.53 mM EDTA, Corning). MSCs (200 K/per sample) were stained with the following fluorochrome-conjugated antibodies: Indoleamine 2,3-dioxygenase (IDO)-FITC (Invitrogen), CD54 (ICAM)-APC (Miltenyi Biotech) and CD274 (PD-L1)-BV421 (BD Biosciences). Intracellular staining of IDO was completed using the eBiosciences intracellular fixation and permeabilization buffer set, in accordance with manufacturer's instructions. The cells were stained with Ki67-PE and CD3-FITC. Intracellular Ki67 staining was completed using the eBiosciences FoxP3 fixation/permeabilization kit, in accordance with the manufacturer's protocol. Live/Dead cell populations were assessed using Ghost Red 780 viability dye (Tonbo Biosciences). Flow cytometry was performed using an Attune NxT flow cytometer (Thermo Fisher Scientific) and analyzed using FCS Express flow cytometry software.

### Doubling Time

Pre-licensed MSCs were cryo-recovered from liquid nitrogen storage as described above. The cells were washed and plated (4.2K cells/well) to a 6-well plate. The cells were harvested three days later and counted using a hemocytometer. Doubling time (Td) was calculated: Td=T (h)×ln 2/In (final cell count/initial cell count). MSC proliferation was assessed in each culture on day 8 post-isolation.

### MSC-PBMC Co-Culture

Pre-licensed, cryopreserved MSCs were cryo-recovered as described above. The cells were plated to a 96-well plate (6K cells/well) and cultured for 24 hours prior to the addition of healthy allogeneic PBMCs. Cryopreserved PBMCs were slowly thawed by the addition of media to the thawing cells. The cells were washed and directly added to the MSC cultures in a 10:1 ratio of PBMCs to MSCs. The cells were stimulated with 2 ml/ml PHA-L (eBioscience). The co-culture remained undisturbed for a further 4 days. The non-adherent PBMCs were collected, and T-cell proliferation was analyzed by flow cytometry.

### Mouse MSC Isolation and Culture

All animal experiments were approved by the University of Wisconsin-Madison Institutional Animal Care and Use Committee and performed in accordance with the Animal Care and Use Policies of the University of Wisconsin-Madison M006487-R01-A02. Male Balb/c and C57BL/6J mice were used for these studies. Mice were maintained in a 12-hour light/dark cycle and fed Teklad Global 2018 rodent diet (Inotiv), ad libitum. Mice were euthanized by CO₂ asphyxiation. Tissues were dissected and placed in cold HBSS until primary cell isolation/culture.

MSC(AD) were isolated from the epididymal fat pad of 10- to 14-week-old mice, as previously published (Das et al. “STAT5-Smad3 dyad regulates adipogenic plasticity of visceral adipose mesenchymal stromal cells during chronic inflammation,” NPJ Regen Med. 2022, 7, 41). Tissues were minced and digested in 2.0 mg/ml collagen IV (Worthington) for 20 minutes at 37° C. with gentle rotation. The filtered cells were washed and plated to a Falcon T-75 flask containing a-MEM media supplemented with 20% charcoal-stripped FBS, 12.5 mM Glutamax and 1% penicillin/streptomycin.

MSC(BM) were isolated from the femur and tibia of 10- to 14-week-old mice, as previously published (Maridas et al. “Isolation, Culture, and Differentiation of Bone Marrow Stromal Cells and Osteoclast Progenitors from Mice,” J Vis Exp. 2018, 131). The extruded BM preparation was filtered and plated to a Corning T-25 flask containing a-MEM supplemented with 20% charcoal-stripped FBS, 12.5 mM Glutamax and 1% penicillin/streptomycin.

Submandibular MSC(SG) were isolated following the procedure previously published for the isolation of human labial salivary gland MSCs (McCoy et al. “Minor salivary gland mesenchymal stromal cells derived from patients with Sjögren's syndrome deploy intact immune plasticity,” Cytotherapy. 2021, 23, 301-310). The tissue was digested for 40 minutes at 37° C., with gentle rotation. The filtered tissue was plated to a Corning T-25 flask containing DMEM media supplemented with 20% charcoal-stripped FBS, 2.5 mM Glutamax, and 1% penicillin/streptomycin. Media was changed in all cultures every 2-3 days. Cells were passaged when ≤80% confluent.

### Mouse Irradiation and MSC Injection

Mice were irradiated 24 hours prior to MSC injection using an Xstrahl Small Animal Radiation Research Platform (SARRP, Xstrahl, UK). Mice were anesthetized in a chamber with 3-5% isoflurane at 1-2 L/min O₂. Mice were then moved to a bed fitted with a nose cone, bite bar and head cradle within the SARRP and maintained with 1-3% isoflurane for the duration of treatment. Animals were placed on a bed in the ventral recumbent (prone) position with the forelimbs tucked underneath the body to prevent any unintentionally absorbed dose in the paws. Using the MuriPlan software, a Cone-Beam CT image was acquired with the X-ray tube operating at 60 kV and 0.8 mA with aluminum filtration and a protocol was established for administering a total of 15 Gy split between two beams at 90 and −90 degrees to the animals' neck to affect both sides equally. A 10×10 mm fixed collimator was used to target as much of the salivary glands as possible, while sparing other tissues in the region. By using two lateral beams rather than a single vertical beam at 0 degrees, the intent was to direct more energy to the tissue of interest. Delivery of 15 Gy single dose for the salivary gland irradiation was applied by operating at 220 kV and 13 mA with copper filtration. The dose rate was 2.9032 Gy/min. (or 0.048387 Gy/sec) [155 seconds to administer 7.5 Gy per submandibular gland on each side].

Pre-licensed, cryopreserved MSCs were cryo-recovered as described above. MSCs were harvested by trypsinization and labeled (150K cells/injection to be completed) with Vybrant CM-DiI cell labeling solution (Invitrogen) in accordance with manufacturer's protocol. The cells were resuspended in a total volume of 30 microliters of PBS and maintained in the dark until injection. Unilateral injection was completed within 1 hour of labeling.

In preparation for MSC injection, mice received an IP injection of meloxicam (Norbrook, 10 mg/kg) prior to surgery. The animals were anesthetized with isoflurane throughout the procedure. The cervical/neck area was shaved and prepped for surgery with betadine and 70% EtOH. A small incision (1-2 cm) was made in the dermis, mid-line above the submandibular salivary glands. MSCs were injected unilaterally into the animal's right salivary gland using a 0.5 ml U-100 insulin syringe and a 28G1/2 needle. The cells were injected superficially, viewing the needle tip through the incision. The skin incision was closed using Vetbond surgical glue (3M). All animals were monitored for pain and discomfort for 48 hours, post-surgery.

DiI-labeled MSCs were imaged on an IVIS Spectrum imaging system (Perkin Elmer). Mice were anesthetized with isoflurane and live images were captured immediately following surgical administration of labeled MSCs, and 1, 6, and 14 days thereafter. Fluorescent signal was monitored using excitation/emission wavelengths of 580/535 nm, respectively. Radiant efficiency was recorded. Images were analyzed using LiveImaging 4.5 software.

### Salivary Flow Measurement

Mice were placed in a clean cage and fasted for two hours prior to the initiation of the saliva collection. Saliva collection was completed as previously described (Bagavant et al. “A Method for the Measurement of Salivary Gland Function in Mice,” J Vis Exp. 2018, 131). Mice were anesthetized with isoflurane and injected, IP, with 1 mg/kg pilocarpine HCl (Millipore Sigma). Saliva collection was initiated exactly 4 minutes after pilocarpine administration to pre-weighed collection tubes. Collection continued for 15 minutes. Saliva was absorbed with Salivabio children's swabs (Salimetrics). Following collection, the swab/collection tube apparatus was weighed and data recorded as mg saliva collected/(body weight)×(min). Salivary flow was measured prior to radiation treatment and on day 14 post-surgery.

### Histological Analysis

Mouse submandibular salivary gland tissues were fixed in 10% formalin (Fisher Chemical), embedded in paraffin and cut into 5-micron sections. The paraffin-embedded tissue sections were baked at 60 degrees C. for 20 minutes. Deparaffinization/hydration was completed. The sections were stained with Harris hematoxylin solution (Poly Scientific R&D Corp) for 45 seconds and eosin Y solution (1% alcoholic, G Biosciences) for 30 seconds, which was followed by dehydration and coverslipping using Cytoseal XYL (Epredia). The tissues were imaged using an Olympus DP80 microscope and the cellSens software package.

Particle size and thickness of the secretory granular band of the convoluted granular tubules (CGTs) were measured by histological image processing using ImageJ software. Toward this, the color channels of the original image were separated using the H&E2 deconvolution vector. A basic threshold was applied to the pink color channel to segment the secretory granular band. From this segmentation, median secretory granular band thickness was calculated using the Local Thickness application. Average granular particle size was determined from the segmentation using a watershed filter and the Analyze Particles application.

### Statistical Analysis

All applicable data were analyzed by One-way Anova. Statistical analyses were completed using GraphPad Prism 10 software. Data reported as Avg±SEM. P<0.05 was considered statistically significant. RNA sequencing genes were considered differentially expressed for analysis if the adjusted p-value (Benjamin-Hochberg) was <0.05.

### Results

Human MSC(BM), MSC(AD), MSC(SG) Transcriptomes are Largely Similar but have Some Divergent Features

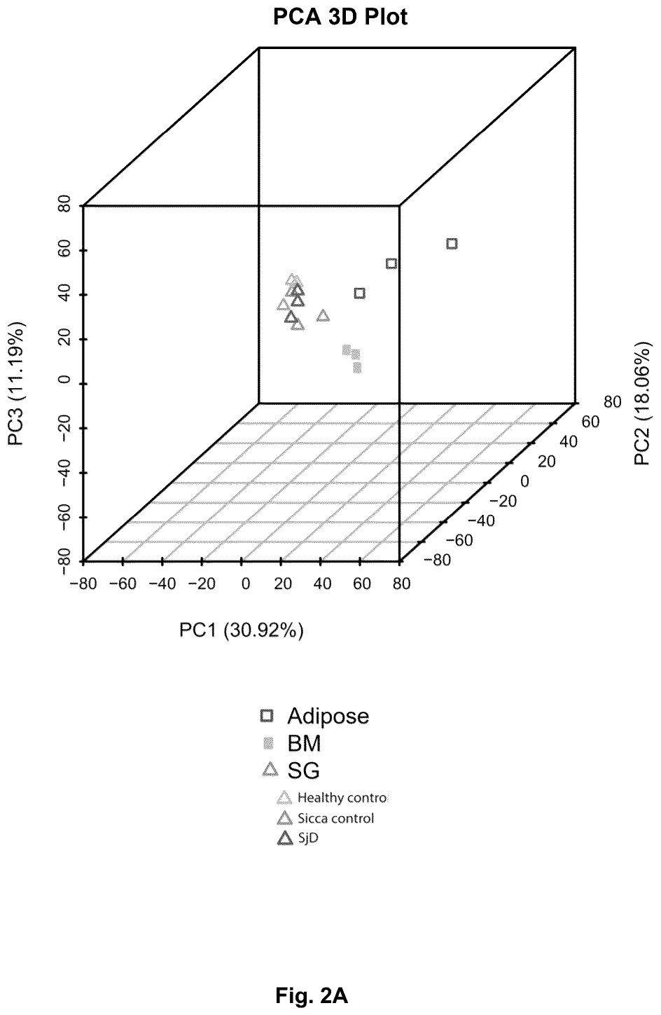

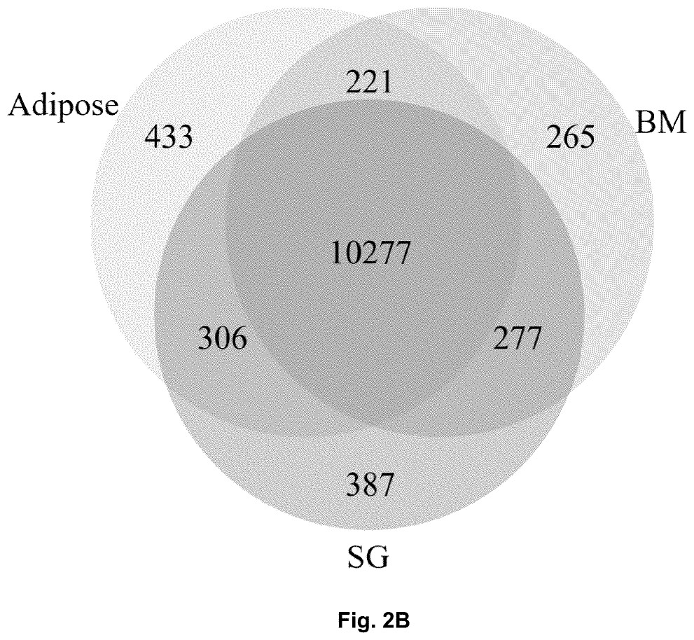

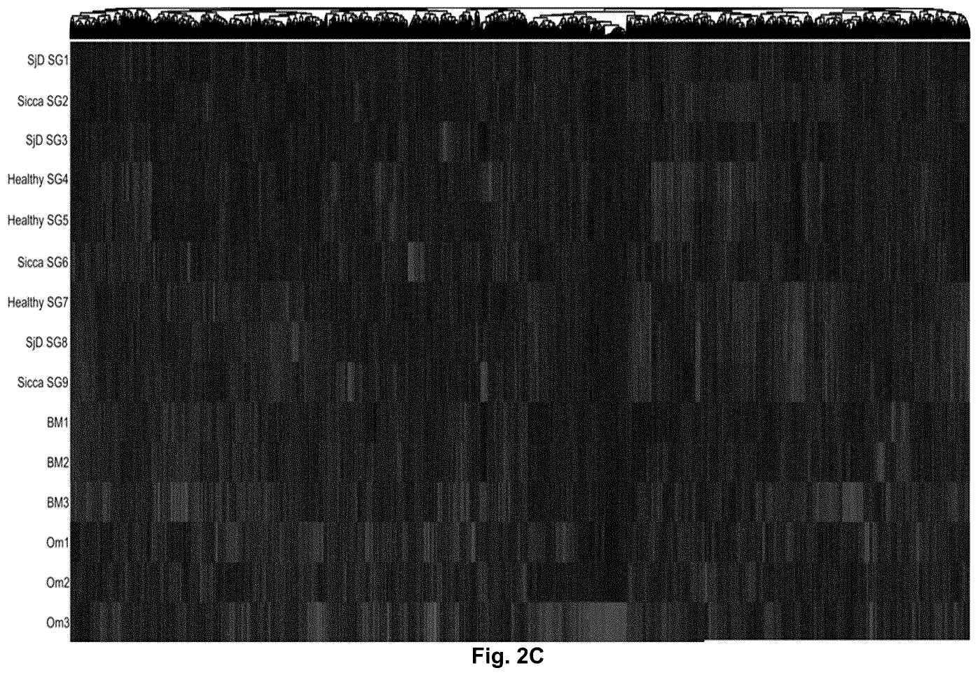

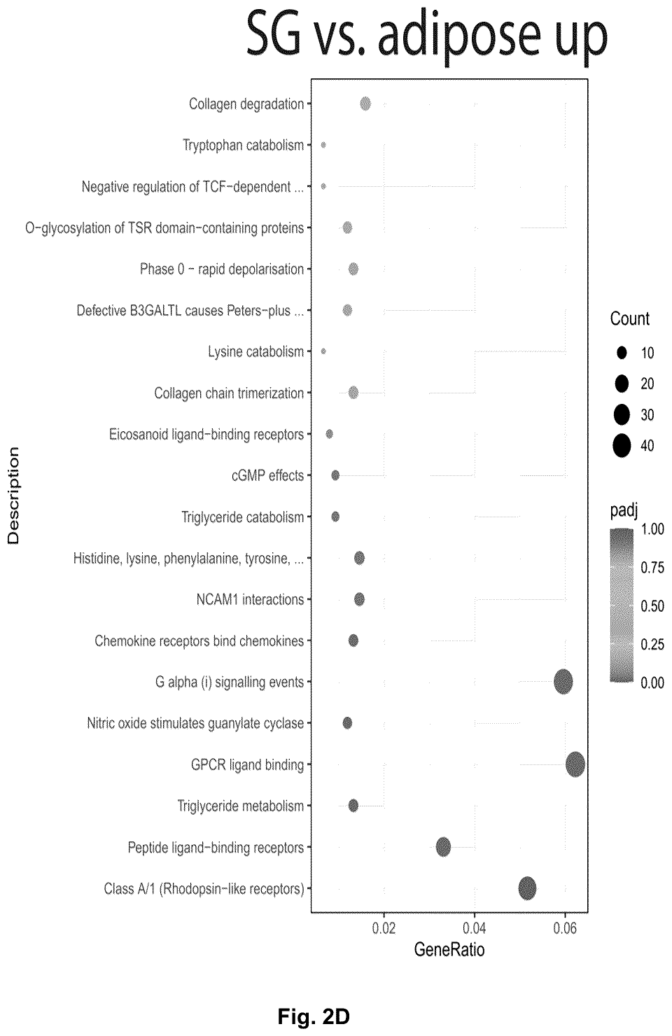

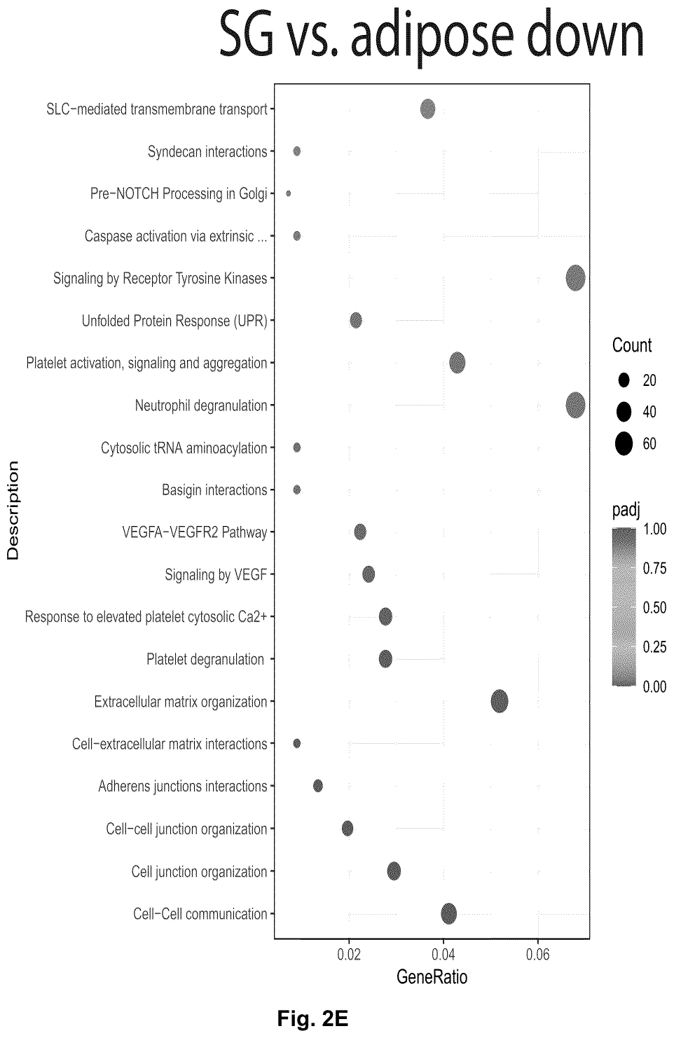

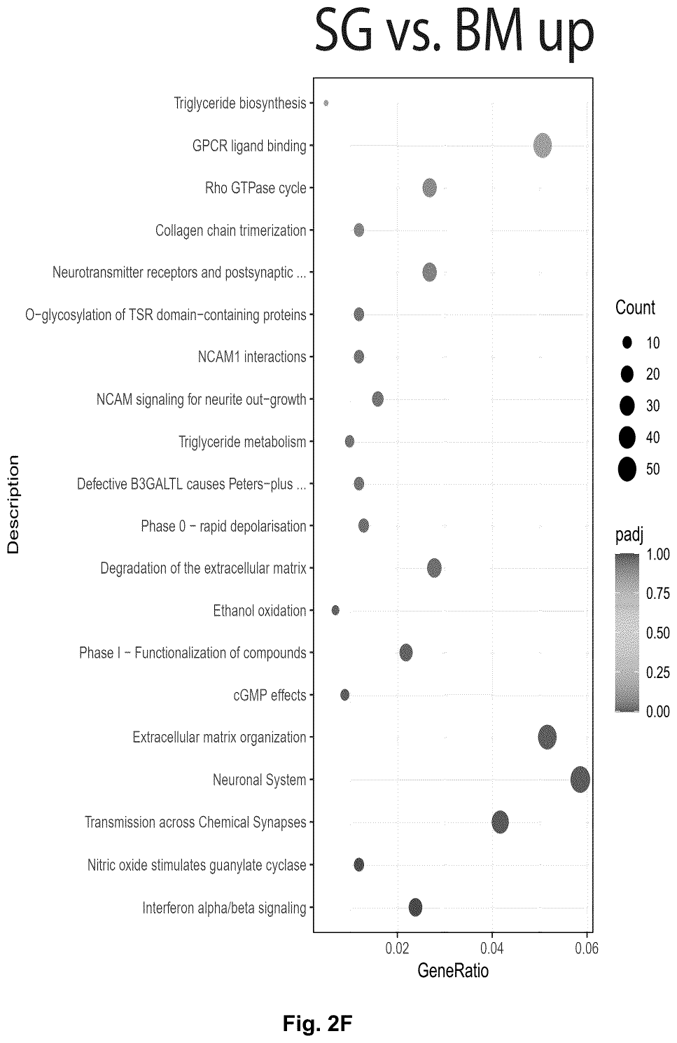

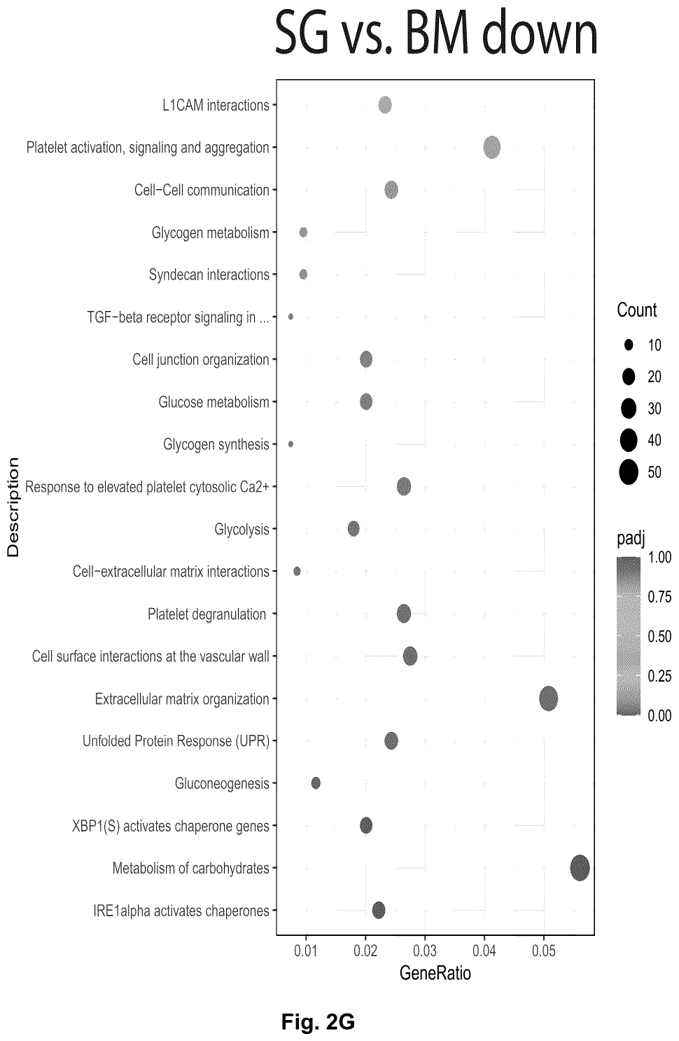

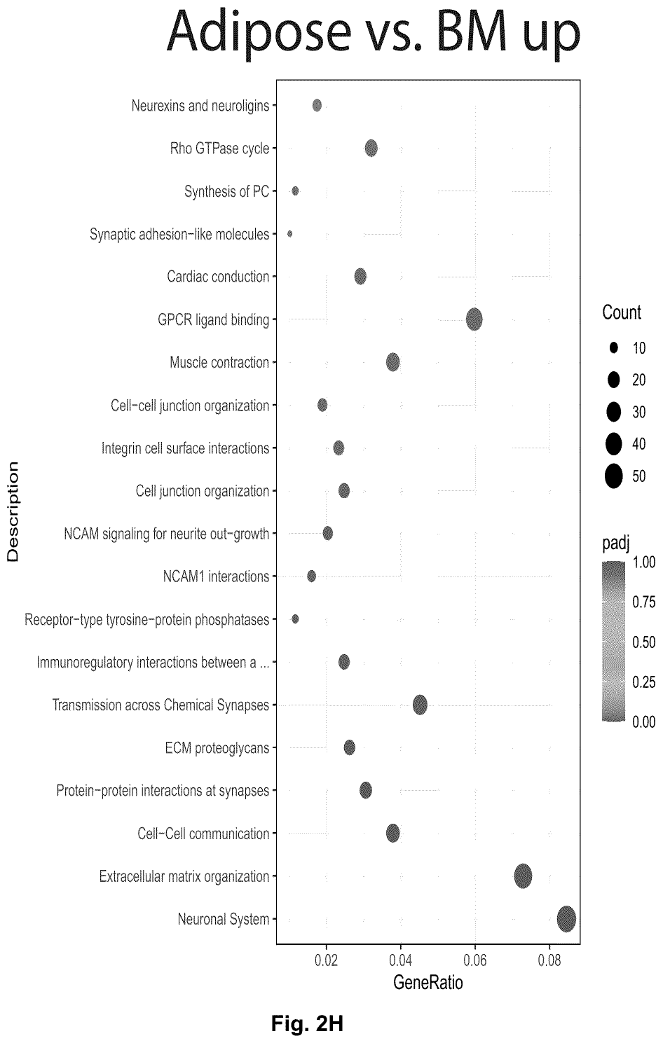

We harvested culture-adapted MSC(BM), MSC(AD), and MSC(SG) and performed bulk RNA sequencing. The transcriptome of each MSC type clustered in a distinct manner (FIG. 2A). Regardless of subject status, MSC(SG) tends to cluster together. Most transcripts are shared between MSC sources (85%), but MSC(AD) has the greatest number of unique transcripts (4%), followed by MSC(SG) (3%), and MSC(BM) (2%) (FIGS. 2B and 2C). The transcripts most significantly increased in MSC(SG) relative to MSC(AD) were related to CLASSA/1 (Rhodopsin-like receptors), a group of G protein-coupled receptors (FIGS. 2D-2I; Table 3).

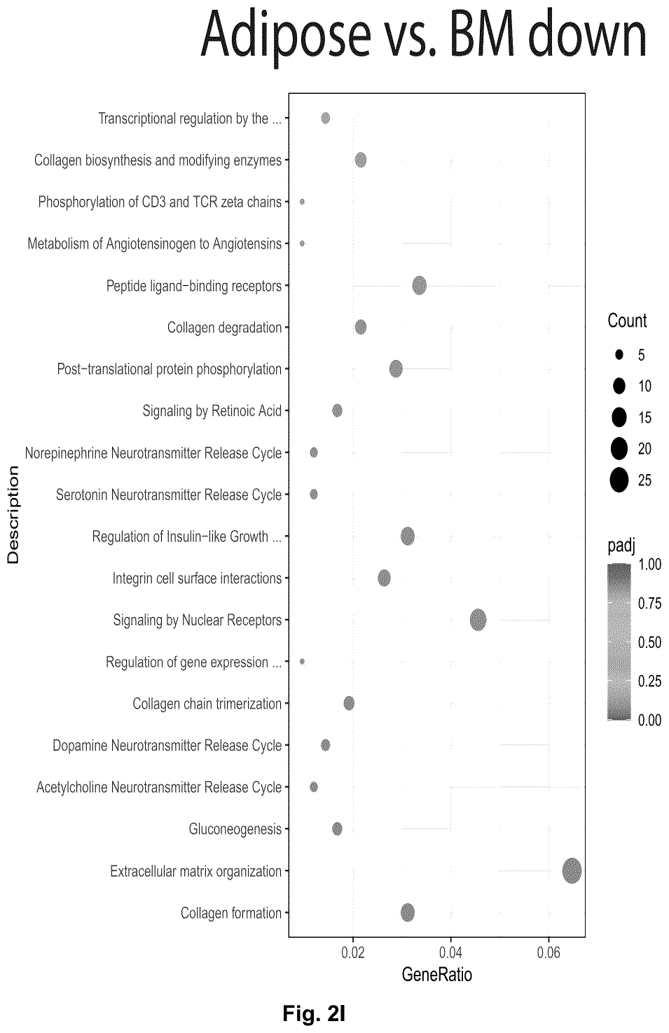

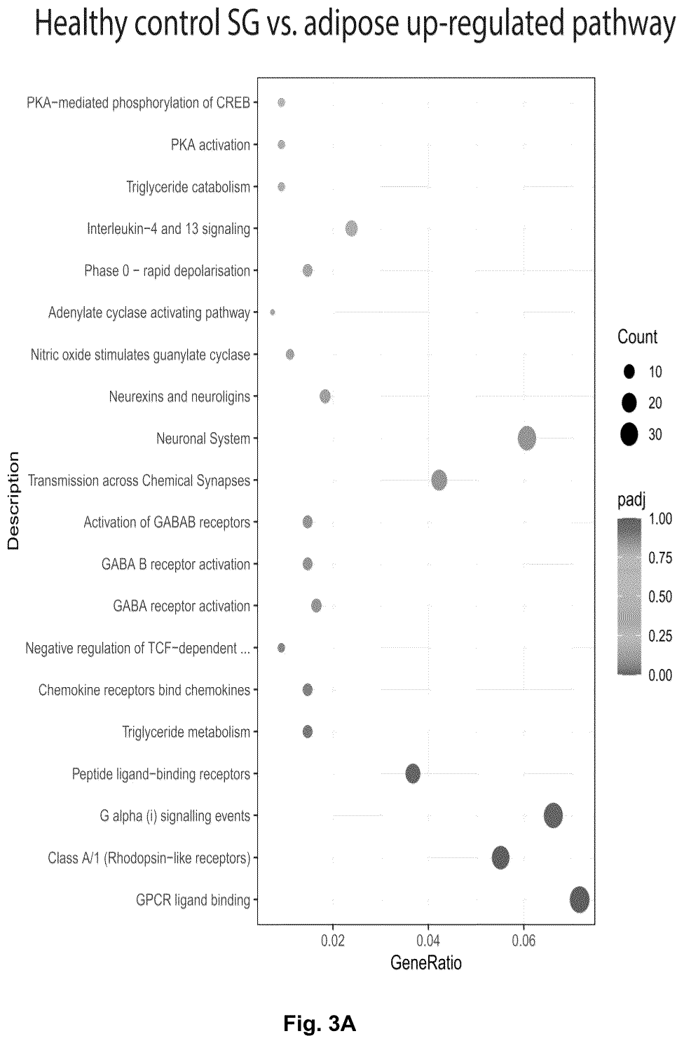

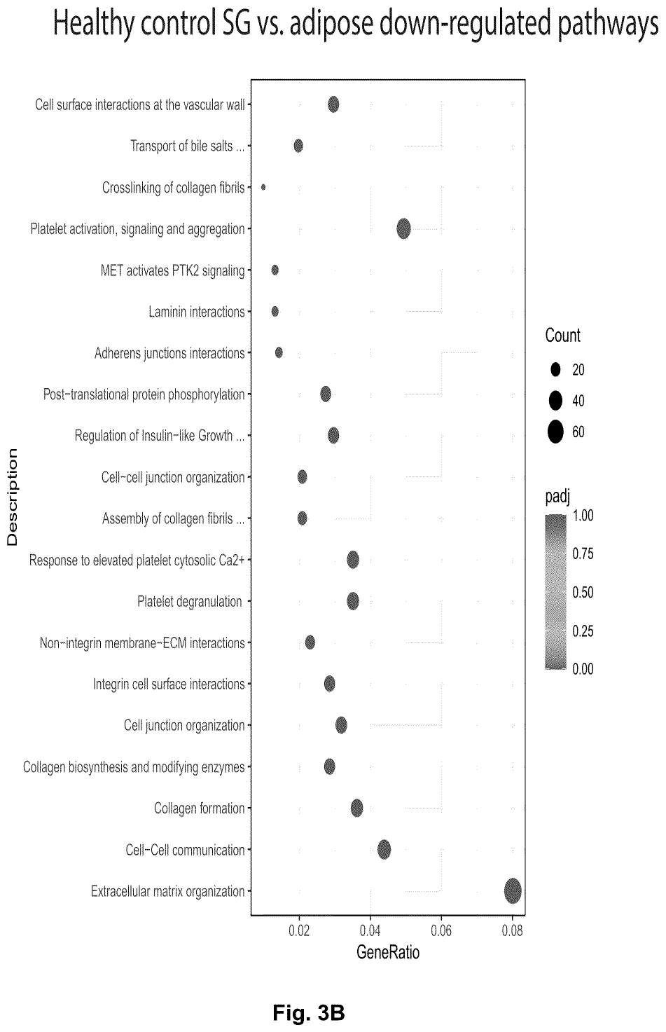

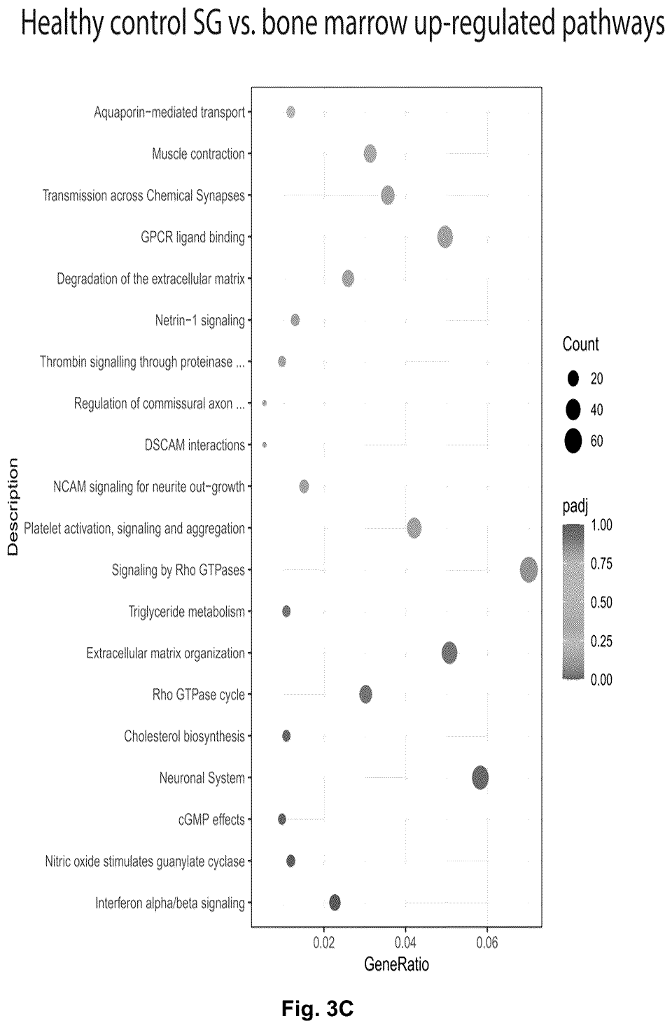

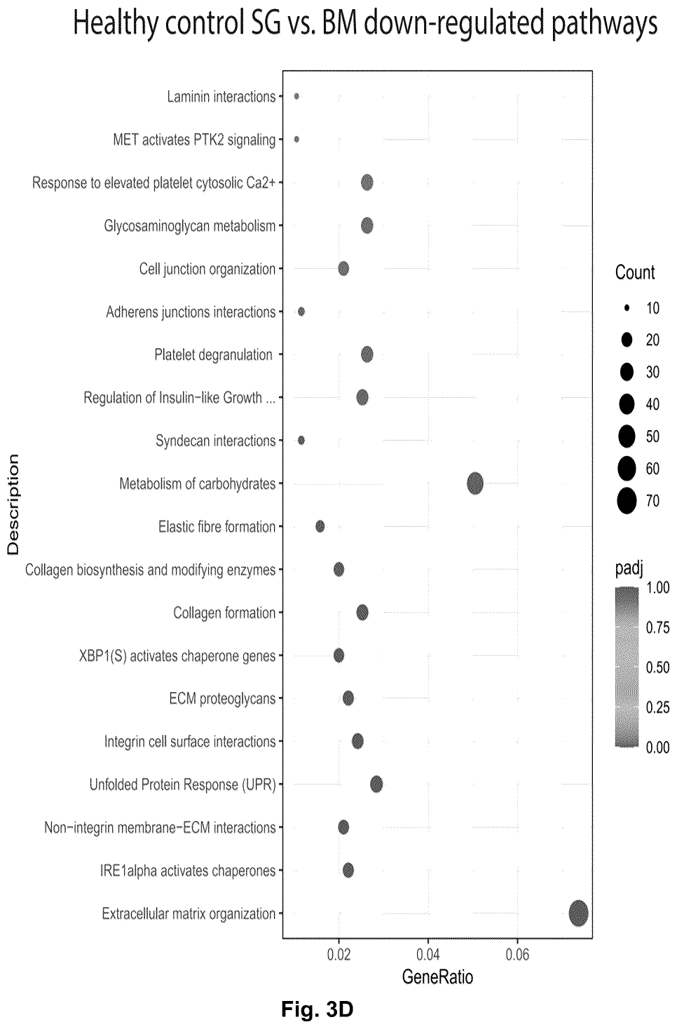

MSC(SG) s have fewer transcripts related to cell-cell communication and extracellular matrix. Compared to MSC(BM), MSC(SG) have upregulated pathways related to interferon alpha/beta signaling and neuronal system related transcripts. Top downregulated pathways in MSC(SG) compared to MSC(BM) include IRE1alpha activation of chaperones. These differences persist when sicca-control and SjD MSCs are excluded from the transcriptomic comparisons (FIG. 3A-3D). Finally, compared to MSC(BM),

MSC(AD) have upregulated transcripts related to the neuronal system. There are no significantly enriched downregulated transcripts.

TABLE 3
Differential expression of reactome pathways between samples.

| | | | | |
|---|---|---|---|---|
| Reactome | | | | |
| ID | Description | padj | geneName | Count |

Nonsicca vs. adipose down

| | | | | |
|---|---|---|---|---|
| R-HSA- | GPCR ligand | 0.00415 | F2RL2/NTSR1/WNT9A/PENK/EDNRA/WNT16/CCK/ | 39 |
| 500792 | binding | | PTGER3/CXCL5/CCR1/ADRA1D/APLN/ADCYAP1R1/ | |
| | | | GNG2/CXCL12/PTGER2/BDKRB2/CXCL11/DRD2/ | |
| | | | WNT5A/FZD1/PTGFR/CXCL3/CCKAR/ADM/EDNRB/ | |
| | | | CXCL10/ACKR4/FZD7/FZD5/LPAR3/ADRB2/MCHR1/ | |
| | | | C5/HCAR1/CCL5/GPER1/PLPPR4/S1PR3 | |
| R-HSA- | Class A/1 | 0.00415 | F2RL2/NTSR1/PENK/EDNRA/CCK/PTGER3/CXCL5/ | 30 |
| 373076 | (Rhodopsin-like | | CCR1/ADRA1D/APLN/CXCL12/PTGER2/BDKRB2/CXCL11/ | |
| | receptors) | | DRD2/PTGFR/CXCL3/CCKAR/EDNRB/CXCL10/ | |
| | | | ACKR4/LPAR3/ADRB2/MCHR1/C5/HCAR1/CCL5/ | |
| | | | GPER1/PLPPR4/S1PR3 | |
| R-HSA- | G alpha (i) | 0.00989 | ADCY4/ADCY1/PENK/PDE1B/PTGER3/CXCL5/CCR1/ | 36 |
| 418594 | signalling | | RGS9/APLN/RGS7/GNG2/CXCL12/SDC1/AKR1C3/ | |
| | events | | BDKRB2/CXCL11/DRD2/PDE4B/RGS20/ADCY2/ | |
| | | | CXCL3/ADCY8/AKR1C1/PPP1R1B/CXCL10/LPAR3/ | |
| | | | MCHR1/C5/HCAR1/CCL5/GPER1/RGS10/CALM3/SDC2/ | |
| | | | CAMKMT/S1PR3 | |
| R-HSA- | Peptide ligand- | 0.00989 | F2RL2/NTSR1/PENK/EDNRA/CCK/CXCL5/CCR1/APLN/ | 20 |
| 375276 | binding receptors | | CXCL12/BDKRB2/CXCL11/CXCL3/CCKAR/EDNRB/ | |
| | | | CXCL10/ACKR4/MCHR1/C5/CCL5/GPER1 | |
| R-HSA- | Triglyceride | 0.03564 | DGAT2/FABP4/AGMO/GPD2/LPIN1/FABP3/CAV1/ | 8 |
| 8979227 | metabolism | | FABP5 | |

Nonsicca vs. adipose down

| | | | | |
|---|---|---|---|---|
| R-HSA- | Extracellular | 2.58E−12 | ICAM1/COL4A1/PDGFA/ITGA11/F11R/ITGB5/ITGB8/ | 73 |
| 1474244 | matrix | | SPARC/ICAM5/COL12A1/COL4A2/P4HA3/COL13A1/ | |
| | organization | | TGFB1/SERPINE1/P4HA2/ITGA2/DDR1/AGRN/ADAM19/ | |
| | | | ITGAV/COL1A1/P4HA1/LAMA1/SERPINH1/MFAP3/ | |
| | | | COL11A1/PXDN/PLOD2/MFAP2/MMP15/ITGA3/ | |
| | | | CDH1/MMP10/BMP4/COL5A2/LAMC2/LOXLA/ACTN1/ | |
| | | | COL18A1/FMOD/COL5A1/TGFB2/LOXL2/CD151/ITGA1/ | |
| | | | COL3A1/BMP1/MMP14/DMD/PLOD1/LOX/SPOCK3/ | |
| | | | P3H1/COL4A5/ADAMTS3/ITGA5/KDR/ADAMTS2/ | |
| | | | PLOD3/COLGALT1/COL22A1/FBLN2/IBSP/BMP2/ | |
| | | | P3H2/TIMP1/LAMB1/SPP1/ITGA6/COL27A1/ | |
| | | | SH3PXD2A/ADAM15 | |
| R-HSA- | Cell-Cell | 7.71E−10 | SKAP2/CDH2/CDH13/F11R/NECTIN2/CLDN1/SIRPB1/ | 40 |
| 1500931 | communication | | FBLIM1/JUP/PRKCI/CDH4/SDK2/NECTIN4/SIRPA/ | |
| | | | CDH8/CDH1/PTK2/LIMS2/PVR/LAMC2/ACTN1/NCK2/ | |
| | | | PARVA/CDH10/CD2AP/KIRREL2/CDH5/CD151/NECTIN1/ | |
| | | | PTK2B/FLNA/VASP/CLDN14/ACTN2/CDH11/PTPN6/ | |
| | | | PARD3/SFTPD/LIMS1/ITGA6 | |
| R-HSA- | Collagen | 7.71E−10 | COL4A1/COL12A1/COL4A2/P4HA3/COL13A1/P4HA2/ | 33 |
| 1474290 | formation | | COL1A1/P4HA1/SERPINH1/COL11A1/PXDN/PLOD2/ | |
| | | | COL5A2/LAMC2/LOXL4/COL18A1/COL5A1/LOXL2/ | |
| | | | CD151/COL3A1/BMP1/PLOD1/LOX/P3H1/COL4A5/ | |
| | | | ADAMTS3/ADAMTS2/PLOD3/COLGALT1/COL22A1/ | |
| | | | P3H2/ITGA6/COL27A1 | |
| R-HSA- | Collagen | 5.82E−08 | COL4A1/COL12A1/COL4A2/P4HA3/COL13A1/P4HA2/ | 26 |
| 1650814 | biosynthesis and | | COL1A1/P4HA1/SERPINH1/COL11A1/PLOD2/COL5A2/ | |
| | modifying | | COL18A1/COL5A1/COL3A1/BMP1/PLOD1/P3H1/ | |
| | enzymes | | COL4A5/ADAMTS3/ADAMTS2/PLOD3/COLGALT1/ | |
| | | | COL22A1/P3H2/COL27A1 | |
| R-HSA- | Cell junction | 2.66E−07 | CDH2/CDH13/F11R/NECTIN2/CLDN1/FBLIM1/JUP/ | 29 |
| 446728 | organization | | PRKCI/CDH4/SDK2/NECTIN4/CDH8/CDH1/LIMS2/PVR/ | |
| | | | LAMC2/ACTN1/PARVA/CDH10/CDH5/CD151/ | |
| | | | NECTIN1/FLNA/VASP/CLDN14/CDH11/PARD3/LIMS1/ | |
| | | | ITGA6 | |
| R-HSA- | Integrin cell | 4.95E−06 | ICAM1/COL4A1/ITGA11/F11R/ITGB5/ITGB8/ICAM5/ | 26 |
| 216083 | surface | | COL4A2/COL13A1/ITGA2/AGRNITGAV/COL1A1/ITGA3/ | |
| | interactions | | CDH1/COL5A2/COL18A1/COL5A1/ITGA1/COL3A1/ | |
| | | | COL4A5/ITGA5/KDR/IBSP/SPP1/ITGA6 | |
| R-HSA- | Non-integrin | 1.42E−05 | COL4A1/PDGFA/ITGB5/COL4A2/TGFB1/ITGA2/DDR1/ | 21 |
| 3000171 | membrane-ECM | | AGRN/ITGAV/COL1A1/LAMA1/COL11A1/COL5A2/ | |
| | interactions | | LAMC2/ACTN1/COL5A1/COL3A1/DMD/COL4A5/ | |
| | | | LAMB1/ITGA6 | |
| R-HSA- | Platelet | 1.43E−05 | RAB27B/PDGFA/STXBP2/TOR4A/SPARC/TGFB1/ | 32 |
| 114608 | degranulation | | SERPINE1/APOA1/EGF/RARRES2/SRGN/LHFPL2/QSOX1/ | |
| | | | BRPF3/TLN1/ITIH3/VEGFC/ACTN1/CD9/TGFB2/ | |
| | | | LGALS3BP/FLNA/SERPINA1/PSAP/GTPBP2/CAP1/ | |
| | | | HSPA5/TAGLN2/ACTN2/TIMP1/TUBA4A/SELP | |
| R-HSA- | Response to | 3.63E−05 | RAB27B/PDGFA/STXBP2/TOR4A/SPARC/TGFB1/ | 32 |
| 76005 | elevated platelet | | SERPINE1/APOA1/EGF/RARRES2/SRGN/LHFPL2/QSOX1/ | |
| | cytosolic Ca2+ | | BRPF3/TLN1/ITIH3/VEGFC/ACTN1/CD9/TGFB2/ | |
| | | | LGALS3BP/FLNA/SERPINA1/PSAP/GTPBP2/CAP1/ | |
| | | | HSPA5/TAGLN2/ACTN2/TIMP1/TUBA4A/SELP | |
| R-HSA- | Assembly of | 0.00018 | COL4A1/COL12A1/COL4A2/COL1A1/COL11A1/ | 19 |
| 2022090 | collagen fibrils | | PXDN/COL5A2/LAMC2/LOXL4/COL18A1/COL5A1/ | |
| | and other | | LOXL2/CD151/COL3A1/BMP1/LOX/COL4A5/ITGA6/ | |
| | multimeric | | COL27A1 | |
| | structures | | | |
| R-HSA- | Cell-cell junction | 0.00021 | CDH2/CDH13/F11R/NECTIN2/CLDN1/JUP/PRKCI/ | 19 |
| 421270 | organization | | CDH4/SDK2/NECTIN4/CDH8/CDH1/PVR/CDH10/CDH5/ | |
| | | | NECTIN1/CLDN14/CDH11/PARD3 | |
| R-HSA- | Regulation of | 0.00041 | IGFBP7/PAPPA2/CDH2/IL6/FSTL3/TMEM132A/MELTF/ | 27 |
| 381426 | Insulin-like | | APOA1/QSOX1/AFP/SERPIND1/IGFBP2/MSLN/STC2/ | |
| | Growth Factor | | BMP4/PCSK9/MFGE8/CKAP4/CYR61/SERPINA1/ | |
| | (IGF) transport | | TGOLN2/TIMP1/DNAJC3/LAMB1/PRSS23/SPP1/C3 | |
| | and uptake by | | | |
| | Insulin-like | | | |
| | Growth Factor | | | |
| | Binding Proteins | | | |
| | (IGFBPs) | | | |
| R-HSA- | Post-translational | 0.00046 | IGFBP7/CDH2/IL6/FSTL3/TMEM132A/MELTF/APOA1/ | 25 |
| 8957275 | protein | | QSOX1/AFP/SERPIND1/MSLN/STC2/BMP4/PCSK9/ | |
| | phosphorylation | | MFGE8/CKAP4/CYR61/SERPINA1/TGOLN2/TIMP1/ | |
| | | | DNAJC3/LAMB1/PRSS23/SPP1/C3 | |
| R-HSA- | Adherens | 0.00046 | CDH2/CDH13/NECTIN2/JUP/CDH4/NECTIN4/CDH8/ | 13 |
| 418990 | junctions | | CDH1/PVR/CDH10/CDH5/NECTIN1/CDH11 | |
| | interactions | | | |
| R-HSA- | Laminin | 0.00111 | COL4A1/COL4A2/ITGA2/ITGAV/LAMA1/ITGA3/ | 12 |
| 3000157 | interactions | | LAMC2/COL18A1/ITGA1/COL4A5/LAMB1/ITGA6 | |
| R-HSA- | MET activates | 0.00111 | ITGA2/COL1A1/LAMA1/COL11A1/ITGA3/PTK2/ | 12 |
| 8874081 | PTK2 signaling | | COL5A2/LAMC2/COL5A1/COL3A1/LAMB1/COL27A1 | |
| R-HSA- | Platelet activation, | 0.00131 | RAC2/RAB27B/PDGFA/STXBP2/TOR4A/SPARC/TGFB1/ | 45 |
| 76002 | signaling and | | SERPINE1/APOA1/BCAR1/DGKQ/EGF/RARRES2/ | |
| | aggregation | | SRGN/COL1A1/LHFPL2/QSOX1/GNG4/BRPF3/PTK2/ | |
| | | | TLN1/ITIH3/VEGFC/ACTN1/DGKH/CD9/TGFB2/GNA12/ | |
| | | | LGALS3BP/FLNA/SERPINA1/ITPR2/PSAP/GTPBP2/ | |
| | | | TPR1/CAP1/LYN/HSPA5/TAGLN2/ACTN2/PTPN6/ | |
| | | | RHOB/TIMP1/TUBA4A/SELP | |
| R-HSA- | Crosslinking of | 0.00132 | COL4A1/COL4A2/COL1A1/PXDN/LOXL4/LOXL2/ | 9 |
| 2243919 | collagen fibrils | | BMP1/LOX/COL4A5 | |
| R-HSA- | Transport of bile | 0.00231 | SLC16A3/SLC22A3/SLC6A9/SLC16A1/SLC39A8/EMB/ | 18 |
| 425366 | salts and organic | | SLC44A2/SLC30A7/RUNX1/SLC39A14/SLC30A5/ | |
| | acids, metal ions | | SLC31A1/SLC30A1/SLC22A1/SLC22A18/SLC39A10/ | |
| | and amine | | SLC41A1/SLC22A2 | |
| | compounds | | | |
| R-HSA- | Cell surface | 0.00231 | SLC7A5/SLC7A11/F11R/SLC16A3/TGFB1/TNFRSF10B/ | 27 |
| 202733 | interactions at the | | SLC3A2/ITGAV/TEK/PROCR/OLR1/SLC7A7/COL1A1/ | |
| | vascular wall | | SLC16A1/SIRPA/ITGA3/TSPAN7/GYPC/GRB7/SLC7A6/ | |
| | | | ATP1B1/CXADR/LYN/ITGA5/PTPN6/ITGA6/SELP | |
| R-HSA- | ECM | 0.00231 | COL4A1/ITGB5/SPARC/COL4A2/TGFB1/SERPINE1/ | 19 |
| 3000178 | proteoglycans | | ITGA2/AGRN/ITGAV/COL1A1/LAMA1/COL5A2/FMOD/ | |
| | | | COL5A1/TGFB2/COL3A1/COL4A5/IBSP/LAMB1 | |
| R-HSA- | Elastic fibre | 0.00249 | ITGB5/ITGB8/TGFB1/ITGAV/MFAP3/MFAP2/BMP4/ | 14 |
| 1566948 | formation | | LOXLA/TGFB2/LOXL2/LOX/ITGA5/FBLN2/BMP2 | |
| R-HSA- | Basigin | 0.0026 | SLC7A5/SLC7A11/SLC16A3/SLC3A2/SLC7A7/ | 10 |
| 210991 | interactions | | SLC16A1/ITGA3/SLC7A6/ATP1B1/ITGA6 | |
| R-HSA- | SLC-mediated | 0.00547 | SLC7A5/SLC7A11/SLC16A3/SLC22A3/SLC45A3/ | 37 |
| 425407 | transmembrane | | SLC6A9/SLC3A2/SLC7A7/SLC16A1/SLC4A3/SLC29A4/ | |
| | transport | | SLC1A4/SLC39A8/SLC2A3/SLC38A1/LCN1/EMB/SLC20A1/ | |
| | | | SLC33A1/SLC44A2/SLC12A4/SLC30A7/SLC1A5/RUNX1/ | |
| | | | SLC7A6/SLC39A14/SLC30A5/SLC35A3/SLC31A1/ | |
| | | | SLC30A1/SLC22A1/SLC22A18/SLC9A1/SLC4A2/ | |
| | | | SLC39A10/SLC41A1/SLC22A2 | |
| R-HSA- | MET promotes | 0.00548 | ITGA2/TNS3/COL1A1/LAMA1/COL11A1/ITGA3/ | 13 |
| 8875878 | cell motility | | PTK2/COL5A2/LAMC2/COL5A1/COL3A1/LAMB1/ | |
| | | | COL27A1 | |
| R-HSA- | Collagen chain | 0.00693 | COL4A1/COL12A1/COL4A2/COL13A1/COL1A1/ | 13 |
| 8948216 | trimerization | | COL11A1/COL5A2/COL18A1/COL5A1/COL3A1/COL4A5/ | |
| | | | COL22A1/COL27A1 | |
| R-HSA- | Syndecan | 0.00716 | ITGB5/TGFB1/ITGA2/ITGAV/COL1A1/COL5A2/ | 10 |
| 3000170 | interactions | | ACTN1/COL5A1/COL3A1/ITGA6 | |
| R-HSA- | Signal regulatory | 0.00726 | SKAP2/SIRPB1/SIRPA/PTK2/PTK2B/PTPN6/SFTPD | 7 |
| 391160 | protein family | | | |
| | interactions | | | |
| R-HSA- | Cytosolic tRNA | 0.01258 | MARS/YARS/WARS/SARS/CARS/GARS/NARS/IARS/ | 9 |
| 379716 | aminoacylation | | TARS | |
| R-HSA- | Neuronal System | 0.02271 | NRXN3/KCNC4/KCNG1/MAOA/PTPRF/PANX2/BCHE/ | 50 |
| 112316 | | | NTRK3/KCNAB1/KCNH5/IL1RAP/TSPOAP1/CACNG8/ | |
| | | | GRIP1/CAMK2A/GLS/KCNK6/SHANK2/KCNK13/ | |
| | | | GNG4/SLC38A1/BEGAIN/TSPAN7/ALDH5A1/COMT/ | |
| | | | AP2A2/GRM1/KCNK1/SLITRK6/KCND3/HCN4/KCNG2/ | |
| | | | ACHE/RPS6KA1/ABAT/LRRC4B/SLC22A1/KCNN4/ | |
| | | | PPFIA1/CACNB3/ADCY5/EPB41L5/KCNA3/KCNQ3/ | |
| | | | ACTN2/SYT1/GABRQ/KCNJ12/SLC22A2/SHANK1 | |
| R-HSA- | Cell-extracellular | 0.04219 | FBLIM1/LIMS2/ACTN1/PARVA/FLNA/VASP/LIMS1 | 7 |
| 446353 | matrix | | | |
| | interactions | | | |
| R-HSA- | Unfolded Protein | 0.04308 | DNAJB9/HERPUD1/XBP1/HYOU1/TPP1/ERNI/EXTL2/ | 20 |
| 381119 | Response(UPR) | | TLN1/ASNS/DNAJB11/CREB3L1/GFPT1/PREB/ATF6/ | |
| | | | DDIT3/HSPA5/ATF4/SYVN1/SRPRB/DNAJC3 | |
| R-HSA- | Signaling by | 0.04308 | TRIB3/JUP/BCAR1/ITGAV/PAK3/NRP2/NOS3/PTK2/ | 21 |
| 194138 | VEGF | | VEGFC/NCK2/AHCYL1/SHC2/CDH5/PTK2B/ITPR2/ | |
| | | | SHB/ITPR1/KDR/AKT3/PRR5/BAIAP2 | |
| R-HSA- | Signaling by | 0.04308 | COL4A1/PDGFA/TRIB3/PLAT/COL4A2/PTPN3/ITGA2/ | 63 |
| 9006934 | Receptor | | JUP/BCAR1/ITGAV/EGF/PAK3/NRP2/TNS3/COL1A1/ | |
| | Tyrosine Kinases | | BDNF/LAMA1/COL11A1/RALA/SHC3/NOS3/ERBB2/ | |
| | | | ITGA3/SPINT1/PTK2/ATP6V0A4/DUSP4/COL5A2/ | |
| | | | LAMC2/RNF41/VEGFC/AP2A2/NCK2/THBS2/GRB7/ | |
| | | | COL5A1/AHCYL1/PCSK6/FGFRL1/SHC2/PTPRJ/CDH5/ | |
| | | | COL3A1/RPS6KA1/PTK2B/ITPR2/TAB2/SHB/ITPR1/ | |
| | | | FGFR3/SPINT2/LYN/COL4A5/KDR/PTPN6/ATP6V0B/ | |
| | | | AKT3/LAMB1/PRR5/BAIAP2/SPP1/COL27A1/CDK5R1 | |
| R-HSA- | ATF6 (ATF6- | 0.04954 | XBP1/ATF6/DDIT3/HSPA5/ATF4 | 5 |
| 381183 | alpha) activates | | | |
| | chaperone genes | | | |
| R-HSA- | Organic | 0.04954 | SLC22A3/RUNX1/SLC22A1/SLC22A18/SLC22A2 | 5 |
| 549132 | cation/anion/ | | | |
| | zwitterion transport | | | |
| R-HSA- | Keratan sulfate | 0.04954 | ST3GAL6/B4GALT3/CHST6/CHST1/FMOD/B4GALT4/ | 8 |
| 2022854 | biosynthesis | | BAGALT1/B3GNT2 | |
| R-HSA- | Amino acid | 0.04954 | SLC7A5/SLC7A11/SLC3A2/SLC7A7/SLC1A4/SLC38A1/ | 8 |
| 352230 | transport across | | SLC1A5/SLC7A6 | |
| | the plasma | | | |
| | membrane | | | |
| R-HSA- | Collagen | 0.04954 | COL4A1/COL12A1/COL4A2/COL13A1/COL1A1/COL11A1/ | 14 |
| 1442490 | degradation | | MMP15/MMP10/COL5A2/COL18A1/COL5A1/ | |
| | | | COL3A1/MMP14/COL4A5 | |
| R-HSA- | IRE1alpha | 0.04954 | DNAJB9/XBP1/HYOU1/TPP1/ERN1/EXTL2/TLN1/ | 14 |
| 381070 | activates | | DNAJB11/GFPT1/PREB/HSPA5/SYVN1/SRPRB/DNAJC3 | |
| | chaperones | | | |

Nonsicca vs. bone marrow up

| | | | | |
|---|---|---|---|---|
| R-HSA- | Interferon | 7.76E−05 | IFIT1/USP18/IFIT3/ISG20/MX1/RSAD2/IFIT2/IFITM1/ | 21 |
| 909733 | alpha/beta | | MX2/IFI27/ISG15/OAS1/SAMHD1/IFI35/STAT1/XAF1/ | |
| | signaling | | IRF4/IFI6/SOCS1/OAS3/SOCS3 | |
| R-HSA- | Nitric oxide | 0.00332 | GUCY1A2/PRKG1/PDE3B/PDE1B/MRVI1/KCNMB4/ | 11 |
| 392154 | stimulates | | PDE9A/GUCY1B1/PDE3A/PRKG2/PDE1A | |
| | guanylate cyclase | | | |
| R-HSA- | cGMP effects | 0.01023 | PRKG1/PDE3B/PDE1B/MRVI1/KCNMB4/PDE9A/PDE3A/ | 9 |
| 418457 | | | PRKG2/PDE1A | |
| R-HSA- | Neuronal System | 0.01554 | EPB41L3/PTPRD/SLC1A2/KCNA1/KCNJ2/CACNB2/ | 54 |
| 112316 | | | NCALD/GRIN2A/CAMK4/RASGRF2/CACNB4/NLGN1/ | |
| | | | KCNJ6/BCHE/DLGAP1/GLUL/GNG2/RPS6KA3/CACNA1A/ | |
| | | | EPB41L5/GNG11/SLC1A7/KCNS2/CPLX1/PLCB1/ | |
| | | | ALDH2/KCNMB4/PPFIBP2/CACNG8/GJC1/GNG4/ | |
| | | | ADCY9/SYT9/PLCB2/SLITRK1/ADCY2/KCNK2/ | |
| | | | SLITRK4/NLGN4X/APBA1/GRIA1/NLGN3/ADCY1/ | |
| | | | UNC13B/GABRB3/CAMK2G/RASGRF1/ARHGEF9/SLITRK5/ | |
| | | | KCNB1/EPB41L2/CALM3/ABCC9/NLGN2 | |
| R-HSA- | Cholesterol | 0.01554 | LSS/ACAT2/TM7SF2/IDI1/MVD/MVK/HSD17B7/ | 10 |
| 191273 | biosynthesis | | HMGCS1/SC5D/EBP | |
| R-HSA- | Rho GTPase | 0.03074 | ARHGAP20/A2M/ARHGAP29/SRGAP3/RASGRF2/ | 28 |
| 194840 | cycle | | ARHGAP21/ARHGAP12/MCF2L/ARAP3/ARHGAP27/ | |
| | | | ARHGAP44/RHOJ/ARHGEF39/ARHGAP6/SRGAP1/ | |
| | | | GNA13/ARHGEF33/RAC3/ARHGEF9/NET1/ARHGAP10/ | |
| | | | ARHGAP19/RACGAP1/PLEKHG5/RHOU/FGD4/ | |
| | | | ARHGAP25/ARHGAP17 | |
| R-HSA- | Extracellular | 0.03074 | MFAP5/LAMA2/A2M/TNXB/FBLN2/COL6A6/ELN/ | 47 |
| 1474244 | matrix | | ADAMTS8/ASPN/PCOLCE2/LAMA5/COL9A3/COL4A5/ | |
| | organization | | SDC1/CAPN5/DCN/MMP16/COL19A1/MFAP4/CD47/ | |
| | | | SCUBE3/COL4A6/LAMB3/PLEC/FBLN5/CAPN2/CTSL/ | |
| | | | CTSS/ITGA4/MATN3/ADAMTS3/LAMC3/COL18A1/ | |
| | | | EMILIN2/SH3PXD2A/VWF/NID1/MMP12/COL22A1/ | |
| | | | ICAM2/CAPN14/MADCAM1/ADAM8/LTBP1/BCAN/ | |
| | | | CAST/FBLN1 | |
| R-HSA- | Triglyceride | 0.03123 | LPIN1/FABP4/DGAT2/CAV1/AGMO/GPD2/FABP3/ | 10 |
| 8979227 | metabolism | | MGLL/PPP1CC/GK | |

Nonsicca vs. bone marrow down

| | | | | |
|---|---|---|---|---|
| R-HSA- | Extracellular | 3.50E−10 | TNC/P4HA1/PLOD2/P4HA3/ITGA11/ITGA3/COL25A1/ | 70 |
| 1474244 | matrix | | PDGFA/COL4A1/TGFB1/COL4A2/COL8A1/ADAM19/ | |
| | organization | | COL27A1/LOXL4/ADAMTS1/LOXL2/COL12A1/ITGB2/ | |
| | | | ACTN1/ITGA1/ITGB8/ITGA5/ITGB5/COL11A1/COMP/ | |
| | | | DMD/VCAM1/BGN/PLOD1/SERPINE1/DDR1/ITGAV/ | |
| | | | MMP13/GDF5/EMILIN1/TGFB2/P4HA2/SERPINH1/ | |
| | | | FN1/ITGAX/LAMB1/FURIN/ICAM1/SPARC/COL5A2/ | |
| | | | IBSP/NTN4/LOX/ADAM12/COL5A1/ITGA2/CTSD/ | |
| | | | ADAMTS2/CD151/ITGB6/NID2/MMP15/LAMA1/SDC3/ | |
| | | | P3H1/P3H2/TIMP1/ITGA7/ADAMTS18/MMP14/LTBP2/ | |
| | | | MMP2/P4HB/TRAPPC4 | |
| R-HSA- | IRE1alpha | 0.00017 | DNAJB9/HYOU1/XBP1/TLN1/HSPA5/EXTL2/SRPRB/ | 21 |
| 381070 | activates | | SSR1/PPP2R5B/SYVN1/YIF1A/ZBTB17/TPP1/SEC61G/ | |
| | chaperones | | ARFGAP1/SEC61B/ERN1/SEC61A2/TSPYL2/DNAJC3/ | |
| | | | SERP1 | |
| R-HSA- | Non-integrin | 0.00021 | TNC/PDGFA/COL4A1/TGFB1/COL4A2/ACTN1/ITGB5/ | 20 |
| 3000171 | membrane-ECM | | COL11A1/DMD/DDR1/ITGAV/FN1/LAMB1/COL5A2/ | |
| | interactions | | NTN4/COL5A1/ITGA2/LAMA1/SDC3/TRAPPC4 | |
| R-HSA- | Unfolded Protein | 0.00039 | DNAJB9/HERPUD1/HYOU1/XBP1/TLN1/HSPA5/EXTL2/ | 27 |
| 381119 | Response(UPR) | | SRPRB/HSP90B1/SSR1/PPP2R5B/SYVN1/IGFBP1/ | |
| | | | YIF1A/ZBTB17/TPP1/SEC61G/ARFGAP1/EXOSC5/ | |
| | | | DCSTAMP/SEC61B/ERN1/SEC61A2/TSPYL2/DDIT3/ | |
| | | | DNAJC3/SERP1 | |
| R-HSA- | Integrin cell | 0.00039 | TNC/ITGA11/ITGA3/COL4A1/COL4A2/COL8A1/ITGB2/ | 23 |
| 216083 | surface | | ITGA1/ITGB8/ITGA5/ITGB5/COMP/VCAMI/ITGAV/ | |
| | interactions | | FN1/ITGAX/ICAM1/COL5A2/IBSP/COL5A1/ITGA2/ | |
| | | | ITGB6/ITGA7 | |
| R-HSA- | ECM | 0.00056 | TNC/COL4A1/TGFB1/COL4A2/ITGB5/COMP/BGN/ | 21 |
| 3000178 | proteoglycans | | SERPINE1/ITGAV/TGFB2/FN1/ITGAX/LAMB1/SPARC/ | |
| | | | COL5A2/IBSP/COL5A1/ITGA2/ITGB6/LAMA1/ITGA7 | |
| R-HSA- | XBP1(S) | 0.00056 | DNAJB9/HYOU1/XBP1/TLN1/EXTL2/SRPRB/SSR1/ | 19 |
| 381038 | activates | | PPP2R5B/SYVN1/YIF1A/ZBTB17/TPP1/SEC61G/ | |
| | chaperone genes | | ARFGAP1/SEC61B/SEC61A2/TSPYL2/DNAJC3/SERP1 | |
| R-HSA- | Collagen | 0.00059 | P4HA1/PLOD2/P4HA3/COL25A1/COL4A1/COL4A2/ | 24 |
| 1474290 | formation | | COL8A1/COL27A1/LOXL4/LOXL2/COL12A1/COL11A1/ | |
| | | | PLOD1/MMP13/P4HA2/SERPINH1/COL5A2/LOX/ | |
| | | | COL5A1/ADAMTS2/CD151/P3H1/P3H2/P4HB | |
| R-HSA- | Collagen | 0.00224 | P4HA1/PLOD2/P4HA3/COL25A1/COL4A1/COL4A2/ | 19 |
| 1650814 | biosynthesis and | | COL8A1/COL27A1/COL12A1/COL11A1/PLOD1/P4HA2/ | |
| | modifying | | SERPINH1/COL5A2/COL5A1/ADAMTS2/P3H1/P3H2/ | |
| | enzymes | | P4HB | |
| R-HSA- | Elastic fibre | 0.00224 | TGFB1/LOXL4/LOXL2/ITGB8/ITGA5/ITGB5/ITGAV/ | 15 |
| 1566948 | formation | | GDF5/EMILIN1/TGFB2/FN1/FURIN/LOX/ITGB6/ | |
| | | | LTBP2 | |
| R-HSA- | Metabolism of | 0.00463 | HAS1/PFKFB4/CHSY3/PPP1R3C/ENO2/SLC2A1/CHST6/ | 48 |
| 71387 | carbohydrates | | PGM1/DSE/GPI/PGK1/CHST3/TPI1/ST3GAL6/CHST11/ | |
| | | | B4GALT1/CHST2/TALDO1/BGN/ALDOC/ENO1/ | |
| | | | PCK2/PKM/GYS1/CHST15/PYGL/ALDOA/MANBA/ | |
| | | | BAGALT3/PFKFB3/PGM2L1/GPC6/PAPSS1/HS3ST1/ | |
| | | | CHST1/GAPDH/CHPF/HYAL3/GALNS/PFKP/EXT2/AKR1B1/ | |
| | | | ST3GAL2/SDC3/HK2/B3GNT2/CHST7/HAS2 | |
| R-HSA- | Syndecan | 0.00463 | TNC/TGFB1/ACTN1/ITGB5/ITGAV/FN1/COL5A2/ | 11 |
| 3000170 | interactions | | COL5A1/ITGA2/SDC3/TRAPPC4 | |
| R-HSA- | Regulation of | 0.01396 | IGFBP7/TNC/STC2/CDH2/IL6/IGFBP2/TMEM132A/ | 24 |
| 381426 | Insulin-like | | QSOX1/IGFBP3/FSTL3/GAS6/PAPPA2/CYR61/HSP90B1/ | |
| | Growth Factor | | EVA1A/SERPIND1/FN1/LAMB1/IGFBP1/CHGB/TIMP1/ | |
| | (IGF) transport | | MMP2/P4HB/DNAJC3 | |
| | and uptake by | | | |
| | Insulin-like | | | |
| | Growth Factor | | | |
| | Binding Proteins | | | |
| | (IGFBPs) | | | |
| R-HSA- | Platelet | 0.01608 | VEGFA/RAB27B/SRGN/PDGFA/TUBA4A/TGFB1/ | 25 |
| 114608 | degranulation | | EGF/VEGFC/ACTN1/QSOX1/TMSB4X/SERPINE1/TLN1/ | |
| | | | HSPA5/GAS6/FLNA/ALDOA/TGFB2/PSAP/FN1/MANF/ | |
| | | | SPARC/BRPF3/LAMP2/TIMP1 | |
| R-HSA- | Adherens | 0.01608 | CDH2/CDH13/CADM1/CADM3/ACTG1/PVR/NECTIN4/ | 11 |
| 418990 | junctions | | CDH4/CDH11/NECTIN2/JUP | |
| | interactions | | | |
| R-HSA- | Cell junction | 0.02045 | CDH2/FBLIM1/ACTN1/CDH13/CLDN1/FLNA/CADM1/ | 20 |
| 446728 | organization | | CADM3/ACTG1/PVR/LIMS1/NECTIN4/CDH4/VASP/ | |
| | | | CD151/CDH11/ARHGEF6/NECTIN2/PRKCI/JUP | |
| R-HSA- | Glycosaminoglycan | 0.02158 | HAS1/CHSY3/CHST6/DSE/CHST3/ST3GAL6/CHST11/ | 25 |
| 1630316 | metabolism | | BAGALT1/CHST2/BGN/CHST15/B4GALT3/GPC6/ | |
| | | | PAPSS1/HS3ST1/CHST1/CHPF/HYAL3/GALNS/EXT2/ | |
| | | | ST3GAL2/SDC3/B3GNT2/CHST7/HAS2 | |
| R-HSA- | Response to | 0.02695 | VEGFA/RAB27B/SRGN/PDGFA/TUBA4A/TGFB1/ | 25 |
| 76005 | elevated platelet | | EGF/VEGFC/ACTN1/QSOX1/TMSB4X/SERPINE1/TLN1/ | |
| | cytosolic Ca2+ | | HSPA5/GAS6/FLNA/ALDOA/TGFB2/PSAP/FN1/MANF/ | |
| | | | SPARC/BRPF3/LAMP2/TIMP1 | |
| R-HSA- | MET activates | 0.03195 | ITGA3/COL27A1/COL11A1/FN1/LAMB1/COL5A2/ | 10 |
| 8874081 | PTK2 signaling | | COL5A1/ITGA2/MET/LAMA1 | |
| R-HSA- | Laminin | 0.03943 | ITGA3/COL4A1/COL4A2/ITGA1/ITGAV/LAMB1/ | 10 |
| 3000157 | interactions | | ITGA2/NID2/LAMA1/ITGA7 | |
| R-HSA- | Post-translational | 0.03943 | IGFBP7/TNC/STC2/CDH2/IL6/TMEM132A/QSOX1/ | 21 |
| 8957275 | protein | | IGFBP3/FSTL3/GAS6/CYR61/HSP90B1/EVA1A/ | |
| | phosphorylation | | SERPIND1/FN1/LAMB1/IGFBP1/CHGB/TIMP1/P4HB/ | |
| | | | DNAJC3 | |

Adipose vs. BM UP

| | | | | |
|---|---|---|---|---|
| R-HSA- | Neuronal System | 5.55E−08 | BCHE/KCNC4/CACNG8/EPB41L5/PTPRD/SLC38A1/ | 58 |
| 112316 | | | NRXN3/GNG4/CACNB4/EPB41L3/DLGAP1/GRIP1/PTPRF/ | |
| | | | KCNK1/PANX2/KCNH5/SLC1A7/KCNJ12/GRIN2D/ | |
| | | | KCNA3/KCNK13/SLC1A2/KCNAB1/AP2A2/SHANK2/ | |
| | | | ADCY9/CACNB3/TSPOAP1/DLG3/NTRK3/ACHE/ | |
| | | | KCNG1/CAMK4/CAMK2G/SYT1/LRRC4B/ALDH2/ACTN2/ | |
| | | | PLCB1/CASK/GRIN2B/CACNB2/KCNN3/KCNA6/ | |
| | | | CACNB1/RPS6KA1/BEGAIN/RTN3/GRM1/GRIP2/ | |
| | | | SLITRK4/TSPAN7/SLITRK5/KCNG2/ADCY5/PTPRS/ | |
| | | | GNG11/SLITRK6 | |
| R-HSA- | Extracellular | 2.05E−06 | FBLN2/TNXB/MFAP5/COL18A1/ICAM1/F11R/COL4A5/ | 50 |
| 1474244 | matrix | | MFAP4/COL13A1/ASPN/ADAMTS3/COL22A1/LAMA5/ | |
| | organization | | COL6A6/AGRN/ICAM5/SH3PXD2A/FMOD/LAMA2/ | |
| | | | CDH1/VWF/PXDN/ITGA2/LTBP4/KDR/TIMP2/BMP4/ | |
| | | | CAPN5/LAMB2/MMP11/DDR1/P4HA2/ADAM10/CASK/ | |
| | | | MMP10/ITGB8/CAPN1/MFAP2/ADAMTS9/LRP4/ | |
| | | | ITGA6/MMP15/DCN/MATN3/NCAMI/COL3A1/ | |
| | | | COL1A1/PTPRS/SPARC/CAPN6 | |
| R-HSA- | Cell-Cell | 0.00013 | F11R/CDH6/PTK2/KIRREL3/FLNC/CDH1/CDH24/ | 26 |
| 1500931 | communication | | PARVA/SDK2/NECTIN2/CDH5/JUP/SIRPB1/SIRPA/LIMS2/ | |
| | | | SPTBN1/ACTN2/CDH8/CLDN1/CASK/KIRREL2/MPP5/ | |
| | | | ITGA6/PRKCI/CDH4/NCK2 | |
| R-HSA- | Protein-protein | 0.00027 | EPB41L5/PTPRD/NRXN3/EPB41L3/DLGAP1/PTPRF/ | 21 |
| 6794362 | interactions at | | GRIN2D/SHANK2/DLG3/NTRK3/SYT1/LRRC4B/CASK/ | |
| | synapses | | GRIN2B/BEGAIN/RTN3/GRM1/SLITRK4/SLITRK5/ | |
| | | | PTPRS/SLITRK6 | |
| R-HSA- | ECM | 0.00067 | TNXB/COL4A5/ASPN/LAMA5/COL6A6/AGRN/FMOD/ | 18 |
| 3000178 | proteoglycans | | LAMA2/ITGA2/LAMB2/LRP4/DCN/MATN3/NCAM1/ | |
| | | | COL3A1/COL1A1/PTPRS/SPARC | |
| R-HSA- | Transmission | 0.00428 | BCHE/CACNG8/SLC38A1/GNG4/CACNB4/GRIP1/ | 31 |
| 112315 | across Chemical | | SLC1A7/KCNJ12/GRIN2D/SLC1A2/AP2A2/ADCY9/ | |
| | Synapses | | CACNB3/TSPOAP1/DLG3/ACHE/CAMK4/CAMK2G/ | |
| | | | SYT1/ALDH2/ACTN2/PLCB1/CASK/GRIN2B/CACNB2/ | |
| | | | CACNB1/RPS6KA1/GRIP2/TSPAN7/ADCY5/GNG11 | |
| R-HSA- | Immunoregulatory | 0.0047 | CD200/ICAM1/CXADR/CLEC2B/ICAM5/CDH1/IFITM1/ | 17 |
| 198933 | interactions | | MICA/MICB/NECTIN2/CD34/C3/CD81/HLA-C/ | |
| | between a | | COL3A1/SIGLEC9/COL1A1 | |
| | Lymphoid and a | | | |
| | non-Lymphoid | | | |
| | cell | | | |
| R-HSA- | Receptor-type | 0.0047 | PTPRD/PTPRF/NTRK3/LRRC4B/SLITRK4/SLITRK5/ | 8 |
| 388844 | tyrosine-protein | | PTPRS/SLITRK6 | |
| | phosphatases | | | |
| R-HSA- | NCAM1 | 0.00913 | COL4A5/CACNB4/COL6A6/AGRN/PRNP/CACNA1H/ | 11 |
| 419037 | interactions | | CACNB3/CACNB2/CACNB1/NCAM1/COL3A1 | |
| R-HSA- | NCAM signaling | 0.01039 | COL4A5/CACNB4/PTK2/COL6A6/AGRN/PRNP/ | 14 |
| 375165 | for neurite out- | | CACNA1H/CACNB3/SPTBN1/SPTA1/CACNB2/CACNB1/ | |
| | growth | | NCAM1/COL3A1 | |
| R-HSA- | Cell junction | 0.01299 | F11R/CDH6/FLNC/CDH1/CDH24/PARVA/SDK2/ | 17 |
| 446728 | organization | | NECTIN2/CDH5/JUP/LIMS2/CDH8/CLDN1/MPP5/ITGA6/ | |
| | | | PRKCI/CDH4 | |
| R-HSA- | Integrin cell | 0.0191 | COL18A1/ICAM1/F11R/COL4A5/COL13A1/COL6A6/ | 16 |
| 216083 | surface | | AGRN/ICAM5/CDH1/VWF/ITGA2/KDR/ITGB8/ITGA6/ | |
| | interactions | | COL3A1/COL1A1 | |
| R-HSA- | Cell-cell junction | 0.0191 | F11R/CDH6/CDH1/CDH24/SDK2/NECTIN2/CDH5/ | 13 |
| 421270 | organization | | JUP/CDH8/CLDN1/MPP5/PRKCI/CDH4 | |
| R-HSA- | Muscle | 0.0191 | CACNG8/NPR1/CACNG7/CACNBA/SCN9A/TNNT1/ | 26 |
| 397014 | contraction | | KCNK1/ATP1B1/DES/DYSF/KCNJ12/KCNK13/KCNIP3/ | |
| | | | CACNB3/ATP2A3/CAMK2G/MYH8/ACTN2/CLIC2/ | |
| | | | CACNB2/SCN1B/GATA4/CACNB1/FKBP1B/TBX5/ | |
| | | | MYH3 | |
| R-HSA- | GPCR ligand | 0.0191 | OXTR/PTGER3/SSTR1/PTGIR/GNG4/HTR2B/ACKR4/ | 41 |
| 500792 | binding | | RAMP1/RAMP2/WNT2/SUCNR1/S1PR5/PTHLH/F2R/ | |
| | | | CD55/HTR1F/GPR4/TBXA2R/WNT3/OPN1SW/ECE1/ | |
| | | | S1PR2/PLPPR4/CCRL2/C3/FPR3/LPAR3/CHRM2/HTR2A/ | |
| | | | OPRL1/HRH1/WNT9A/HTR1B/GRM1/FZD4/FZD6/ | |
| | | | GAL/ADM2/VIPR1/BDKRB2/GNG11 | |
| R-HSA- | Cardiac | 0.01972 | CACNG8/NPR1/CACNG7/CACNB4/SCN9A/KCNK1/ | 20 |
| 5576891 | conduction | | ATP1B1/KCNJ12/KCNK13/KCNIP3/CACNB3/ATP2A3/ | |
| | | | CAMK2G/CLIC2/CACNB2/SCN1B/GATA4/CACNB1/ | |
| | | | FKBP1B/TBX5 | |
| R-HSA- | Synaptic | 0.02555 | PTPRD/PTPRF/GRIN2D/DLG3/GRIN2B/RTN3/PTPRS | 7 |
| 8849932 | adhesion-like | | | |
| | molecules | | | |
| R-HSA- | Synthesis of PC | 0.02555 | BCHE/LPIN3/SLC44A5/ACHE/SLC44A2/STARD10/ | 8 |
| 1483191 | | | LPIN1/PCYT1B | |
| R-HSA- | Rho GTPase | 0.02555 | ARHGAP20/RAC2/ARAP2/ARHGAP25/ARHGEF26/ | 22 |
| 194840 | cycle | | ARHGDIG/ARHGAP27/RHOJ/ARHGEF2/AKAP13/ | |
| | | | ARAP3/ARHGAP23/RAC3/TAGAP/ARHGAP26/ITSN1/ | |
| | | | ARHGAP21/ARHGAP44/FGD4/SOS2/OBSCN/RALBP1 | |
| R-HSA- | Neurexins and | 0.04241 | EPB41L5/NRXN3/EPB41L3/DLGAP1/GRIN2D/ | 12 |
| 6794361 | neuroligins | | SHANK2/DLG3/SYT1/CASK/GRIN2B/BEGAIN/GRM1 | |

### The Trophic Secretome Differs by MSC Source and Cytokine Stimulation Conditions

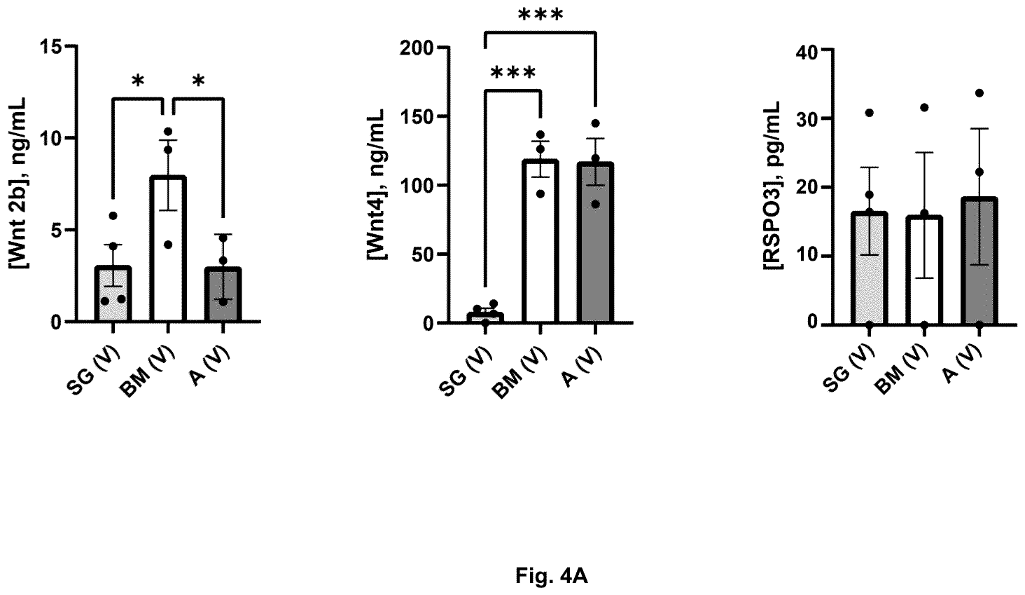

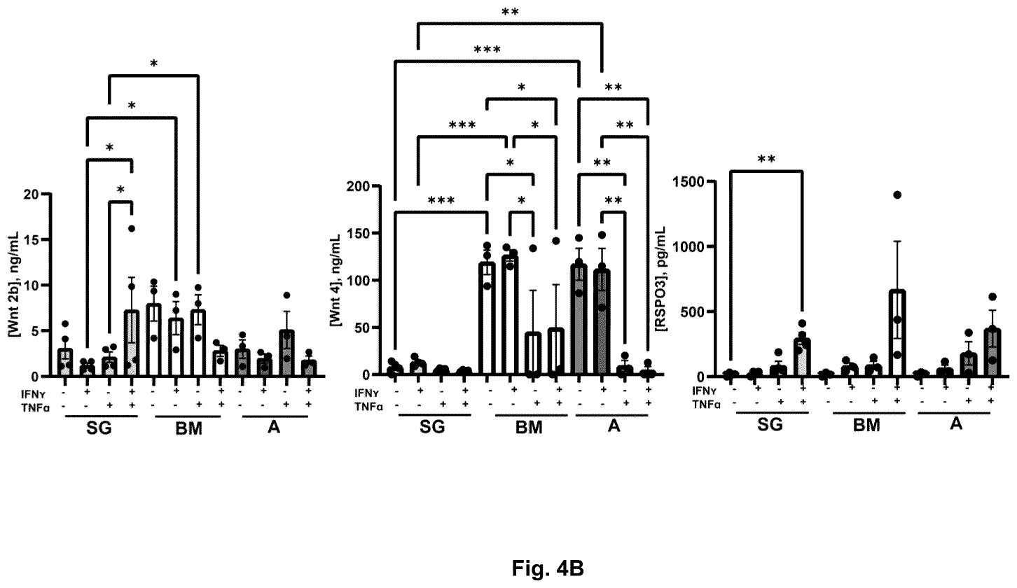

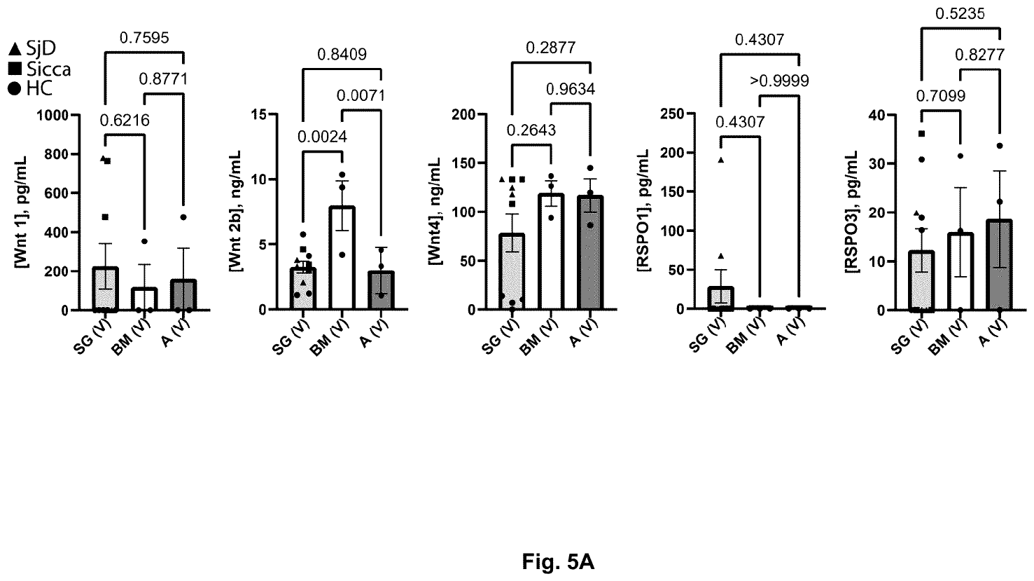

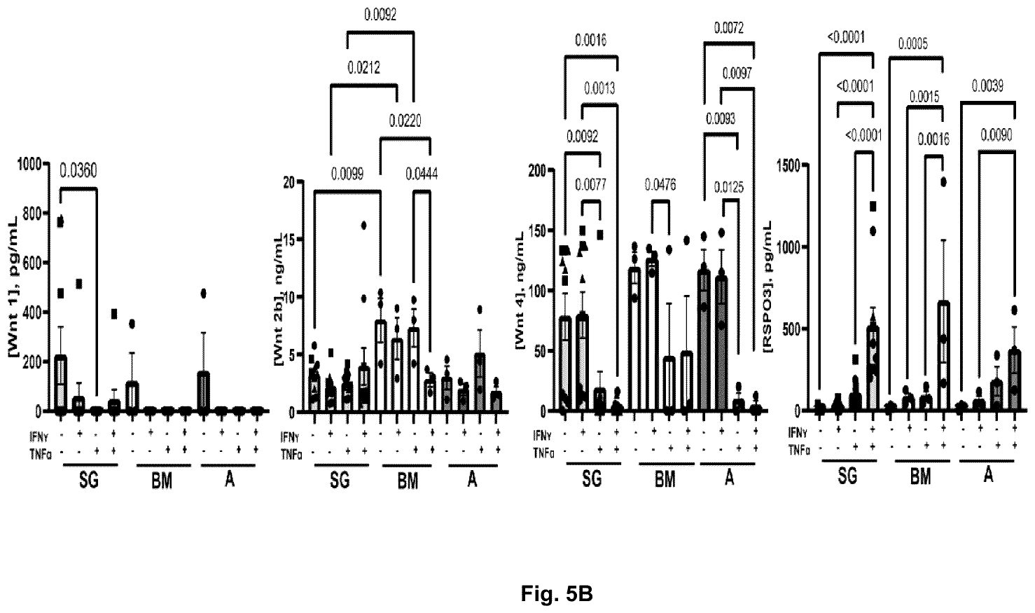

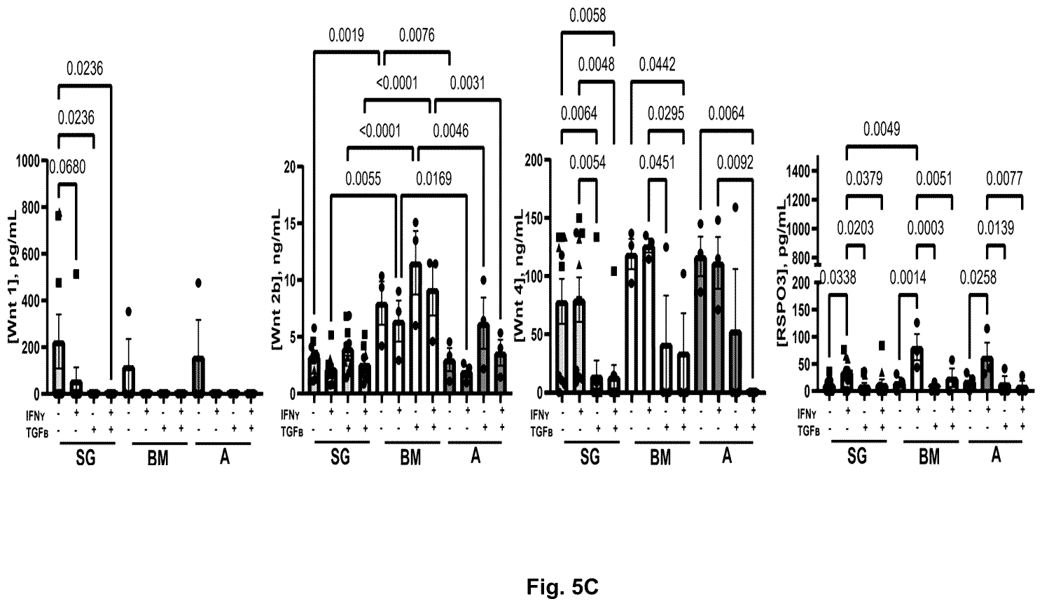

MSCs generate a trophic secretome, capable of supporting local progenitor cell populations. It is unknown whether cytokine pre-licensing affects this trophic secretome. We studied the trophic secretome profile of healthy control culture adapted MSC(SG), MSC(BM), and MSC(AD). Conditioned medium from MSC(BM) has more Wnt2b than MSC(SG) and MSC(AD) (FIG. 4A). MSC(BM) and MSC(AD) produced more Wnt4 than MSC(SG). There was no significant difference between MSC sources in levels of RSPO3 (not shown are results with no significance or measurable protein from Wnt1, GDNF, RSPO1, Wnt3a, and Wnt5a ELISAs). We included additional analyses using MSC(SG) derived from SjD and sicca control subjects. Interestingly, SjD and sicca control subjects had higher levels of Wnt4 than healthy control MSC[SGs] (p<0.0001 for both comparisons) (FIGS. 5A-5B).

IFNγ and TNFα have well established effects on the immunomodulatory behavior of MSCs. (See François et al. “Human MSC suppression correlates with cytokine induction of indoleamine 2,3-dioxygenase and bystander M2 macrophage differentiation,” Mol Ther. 2012, 20, 187-195; English et al. “IFN-gamma and TNF-alpha differentially regulate immunomodulation by murine mesenchymal stem cells,” Immunol Lett. 2007, 110, 91-100; Li et al. “Interferon-γ and tumor necrosis factor-α promote the ability of human placenta-derived mesenchymal stromal cells to express programmed death ligand-2 and induce the differentiation of CD4(+) interleukin-10(+) and CD8(+) interleukin-10(+) Treg subsets,” Cytotherapy. 2015, 17, 1560-1571.) The effect of these cytokines on the MSC secretome and by MSC source, is unknown. We found that Wnt2b significantly increased in MSC(SG) after treatment with IFNγ and TNFα (FIG. 4B) compared to IFNγ-treated, making Wnt2b levels similar between MSC(SG) and MSC(BM). TNFα reduced Wnt4 in all MSC sources with significance in MSC(BM) and MSC(AD). RSPO3 production was synergistically increased by treatment with both IFNγ and TNFα in all MSCs, regardless of source, though only reaching significance in MSC(SG) (FIG. 4B). We additionally investigated the effects of TGFβ pre-licensing on the same secretome markers (FIG. 5C). Wnt4 was suppressed by TGFβ. TGFβ did not clearly enhance any trophic secretome markers.

### Ifnγ and TNFα Combination Synergistically Enhances MSC(SG) Immunomodulatory Capacity

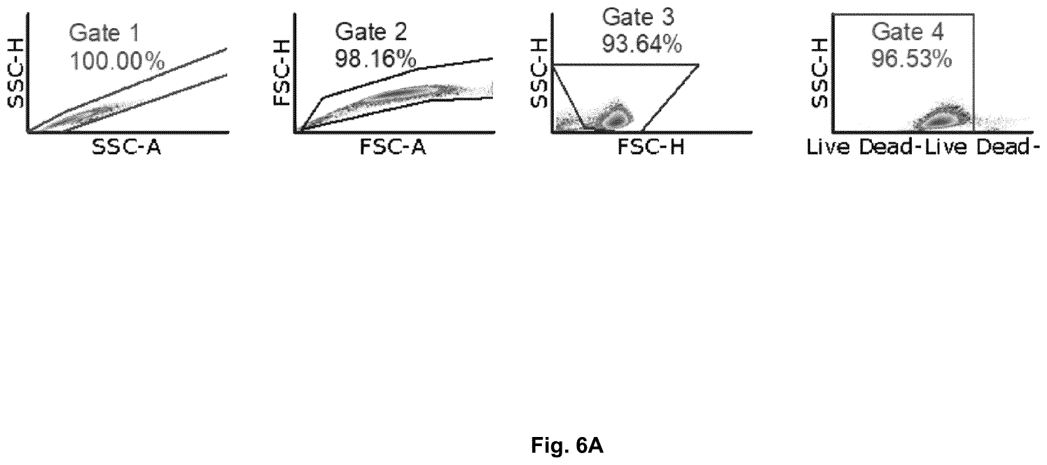

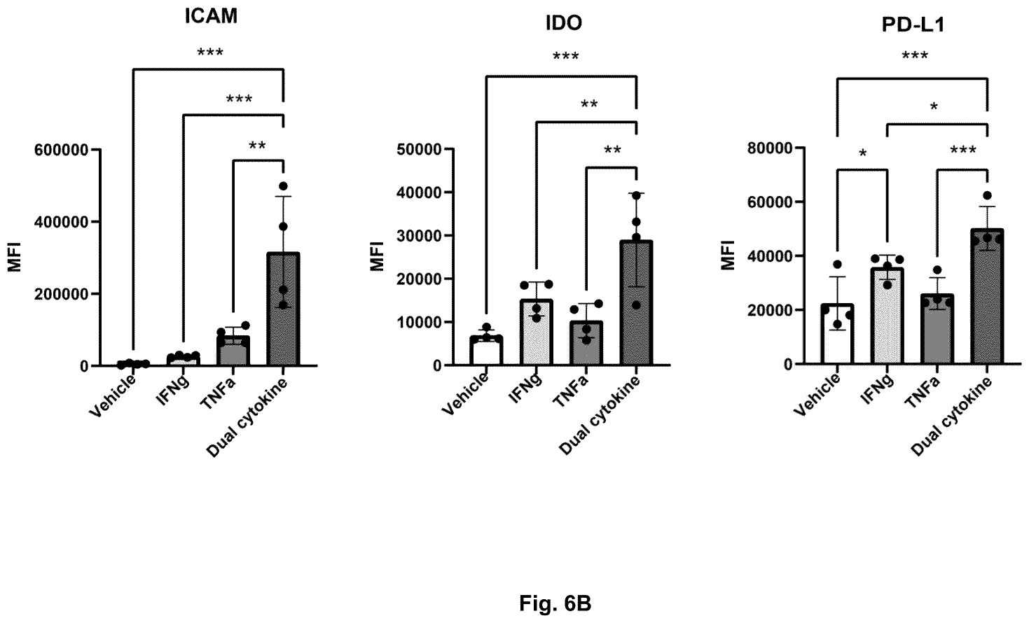

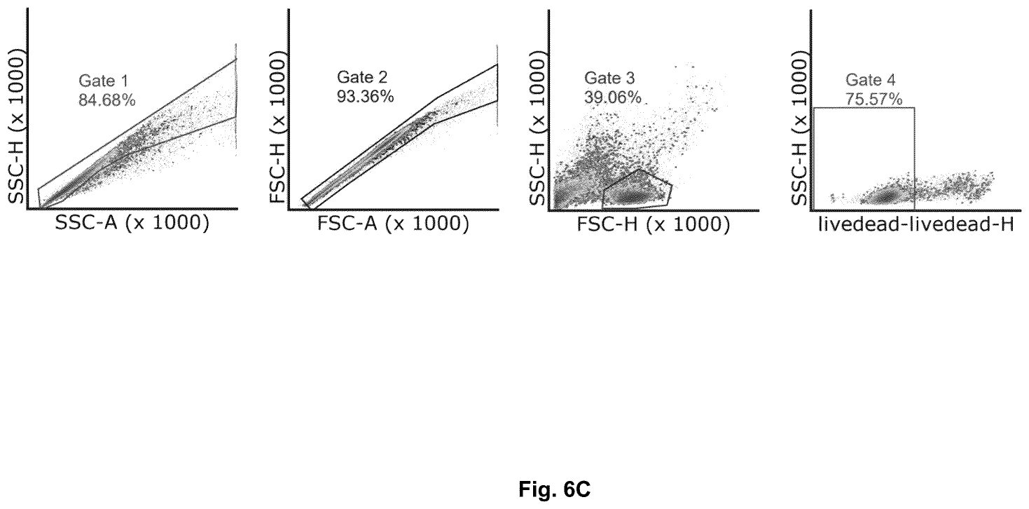

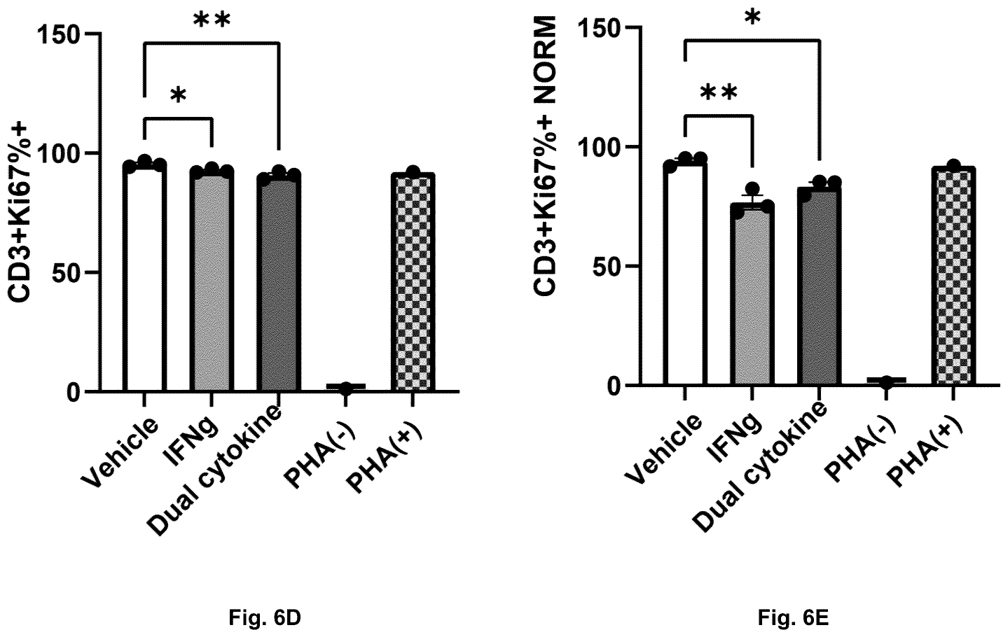

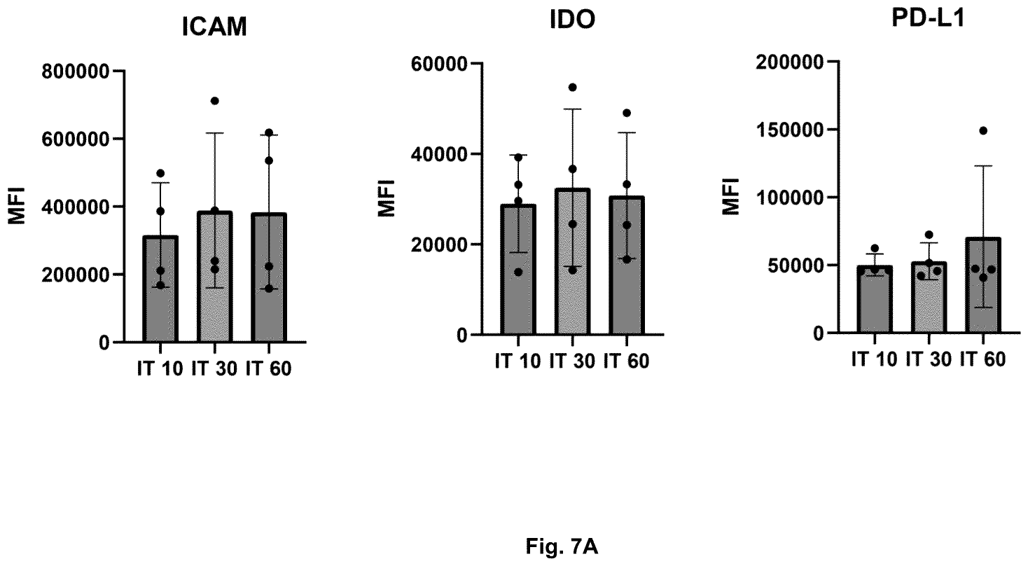

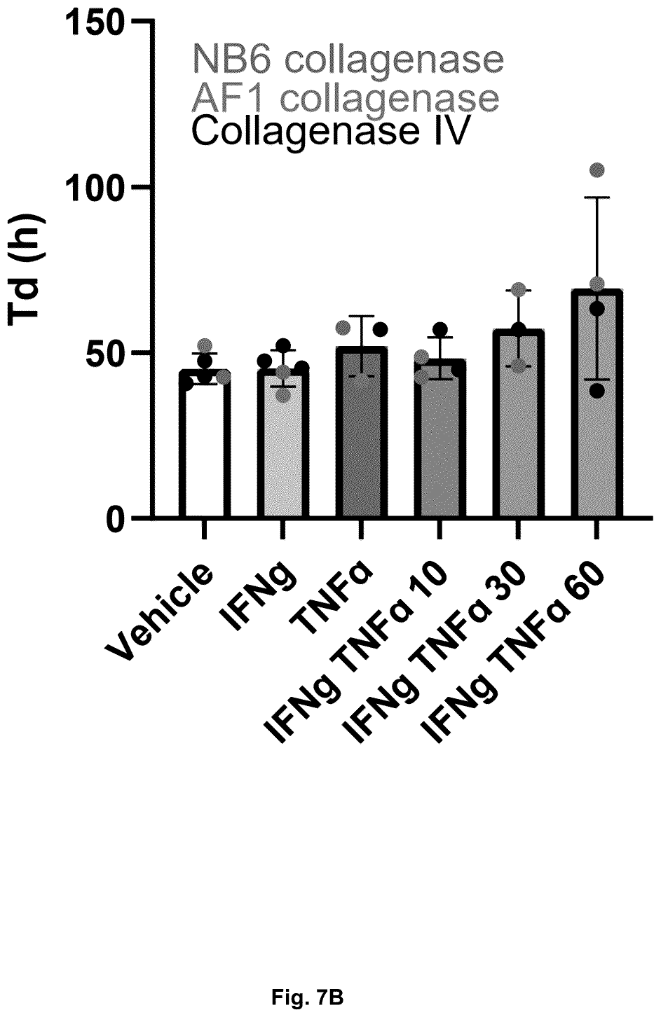

Cryopreservation is critical to the feasibility of using MSCs as therapy; yet cryopreservation impairs MSC immunomodulatory capacity. IFNγ and TNFα have been studied extensively in other traditional MSC sources, but nothing is known of their effect on MSC(SG) immunomodulatory capacity after cryopreservation. We found that after thaw, MSC(SG) pre-licensed with IFNγ and TNFα had higher ICAM1 than vehicle, IFNγ, or TNFα treated conditions (FIGS. 6A-6B). We found that IDO and PD-L1 also were highest in the IFNγ and TNFα condition compared to any of the individual treatment conditions. IFNγ alone significantly increased PD-L1 compared to vehicle condition. We did not find a significant difference in immunomodulatory marker expression between different TNFα doses (FIG. 7A).

We found that cytokine treated MSC(SG) were superior to vehicle treated MSC(SG) regarding their capacity to suppress T-cell proliferation (FIGS. 6C-6D). We corrected for total number of MSCs surviving in co-culture conditions and the results persisted (FIG. 6E).

Given our interest in optimizing MSC(SG) as a potential therapeutic, we sought to determine the effect of IFNγ, TNFα, or both on MSC doubling time. We also compared three different enzyme digestion approaches. We saw no difference in doubling time by cytokine stimulation condition or by enzyme digestion approach (FIG. 7B).

MSC(SG) Pre-Licensed with IFNγ and/or TNFα are Superior to Msc(SG) without Pre-Licensing to Preserve Salivary Gland Function after Radiation in Mice

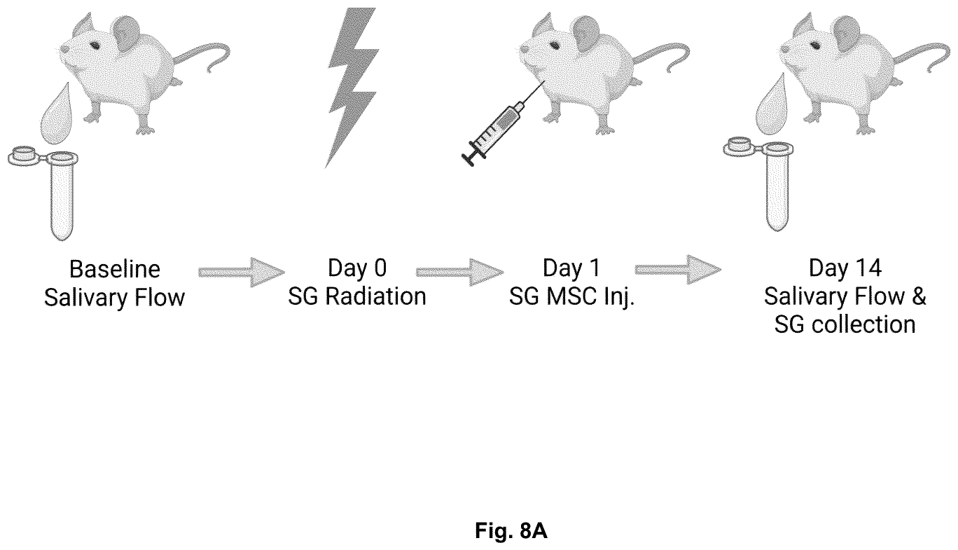

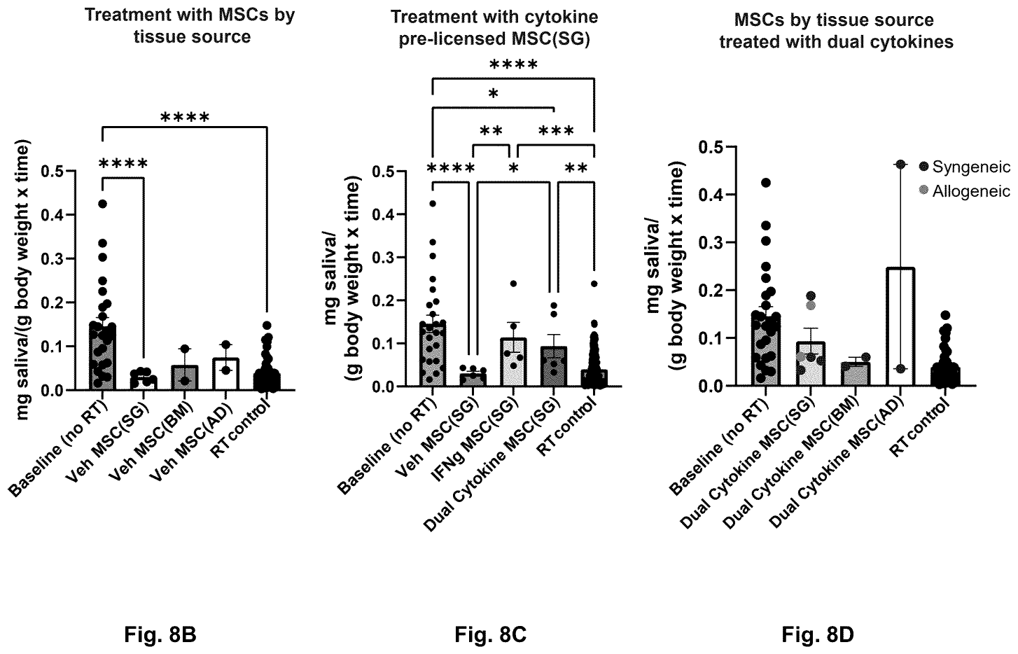

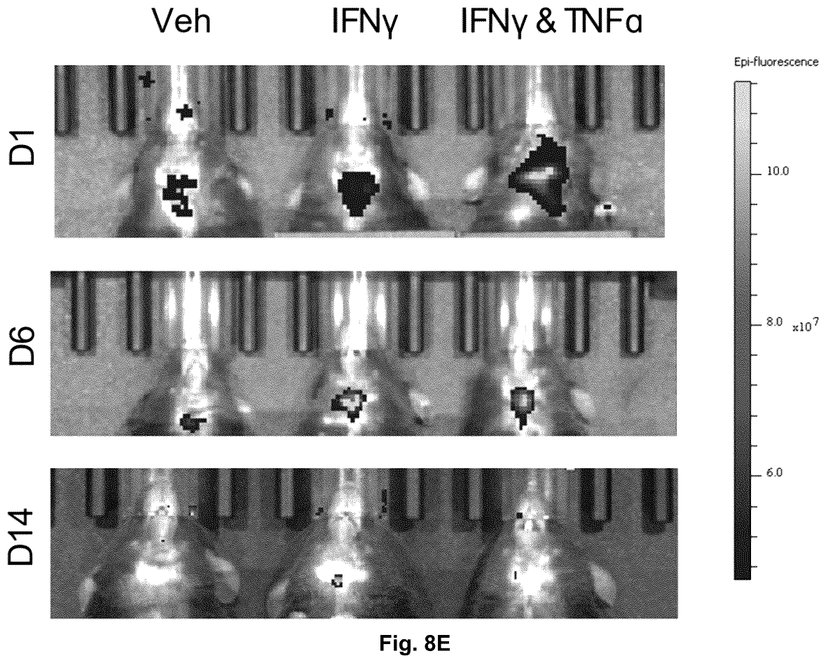

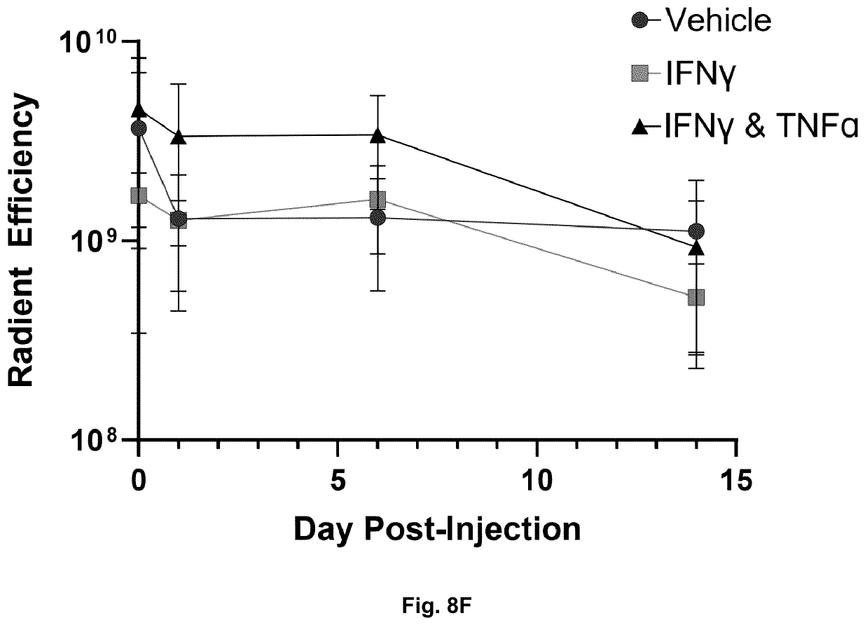

The effect of MSC(SG) and dual cytokine pre-licensing conditions on preservation of salivary flow after radiation therapy is unknown. We studied the effects of MSCs by tissue source and cytokine pre-licensing conditions through MSC injection of mice after radiation therapy (FIG. 8A).

We evaluated whether MSCs from different tissue sources differed in their ability to preserve SG function (FIG. 8B). Mice treated with MSC(SG) lost significantly more salivary flow from baseline compared to MSC(BM) and MSC(AD), which were protective against this loss. Next, we sought to determine if cytokine pre-licensing could optimize the effect of MSC(SG) on preservation of salivary function. MSC(SG) pre-licensed with IFNγ or IFNγ and TNFα (dual cytokine conditions) preserved significantly more salivary flow than untreated MSC(SG) s (FIG. 8C). There was no statistical difference in salivary preservation between MSC(SG) s treated with IFNγ or dual cytokine conditions. Because MSC(SG) preservation of salivary flow was improved after dual cytokine treatment, we next compared dual cytokine pre-licensed MSC(SG) to the dual cytokine pre-licensing in MSCs from other tissue sources. The most robust response to IFNγ and TNFα treatment resulted from MSC(SG) treatment but there was a strong and similar trend seen in MSC(AD) (FIG. 8D). There was no clear difference between dual cytokine pre-licensed MSCs by tissue source. Whether the MSCs were sourced from syngeneic or allogeneic mice appeared to have little effect on the ability to preserve salivary flow.

Although persistence of injected MSCs from other sources is well established, the persistence of locally injected MSC(SG) is unknown. We found that, regardless of cytokine pre-licensing condition, MSC(SG) s persisted at least 14 days in SG tissue (FIGS. 8E and 8F).

MSC(SG) Pre-Licensed with Both IFNγ and TNFα Preserve Salivary Gland Structure Compared to Vehicle or IFNγ Treatment Alone

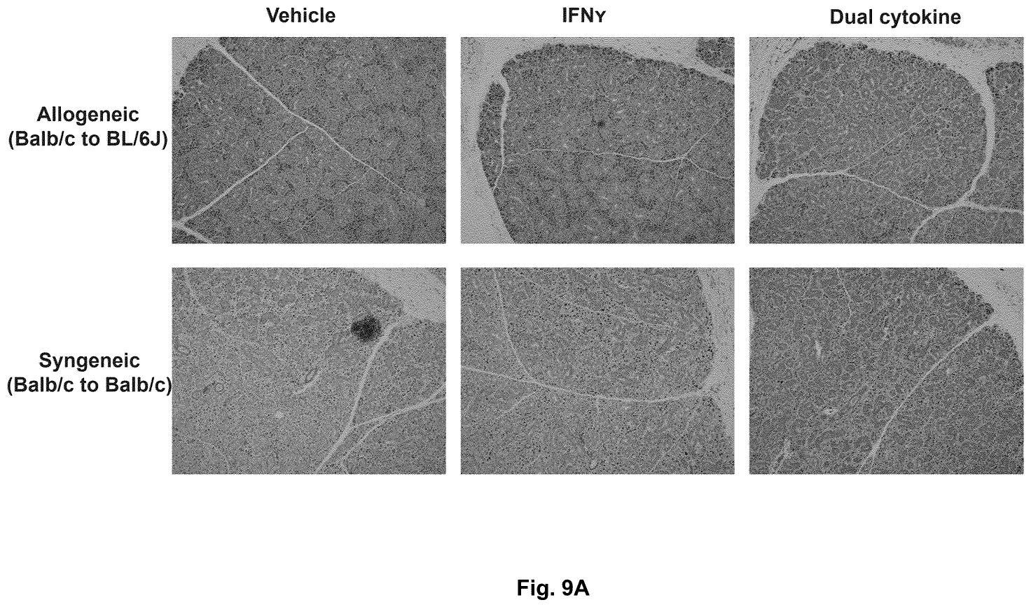

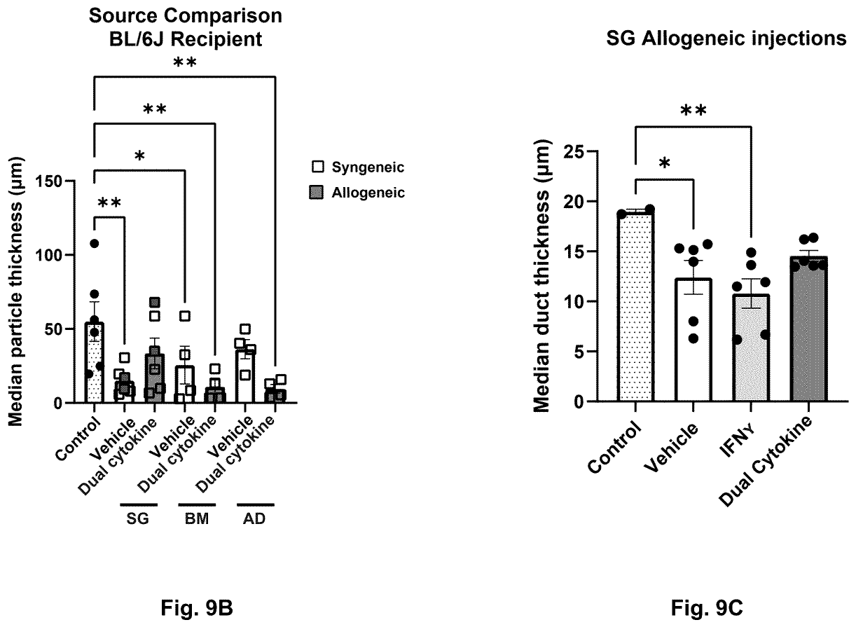

We analyzed the salivary glands of the mice at two weeks post-MSC injection. H&E showed marked protection of salivary gland tissue treated with MSC(SG) that were pre-licensed with both IFNγ and TNFα (FIG. 9A). We saw that dual cytokine treated MSC(SG) preserve duct size (FIG. 9B) in all BL/6J mice. In contrast, IFNγ and TNFα treatment of MSC(BM) and MSC(AD) do not appear to have the same protective effect on duct size. MSC(BM) and MSC(AD) had the greatest protective effect in the vehicle condition. Next, we evaluated the effects of MSC(SG) only when administered allogeneically from Balb/c to BL/6J mice. MSC(SG) treated with both IFNγ and TNFα had greater protective effects than vehicle- and IFNγ-treated MSC(SG) (FIG. 9C).

### Discussion

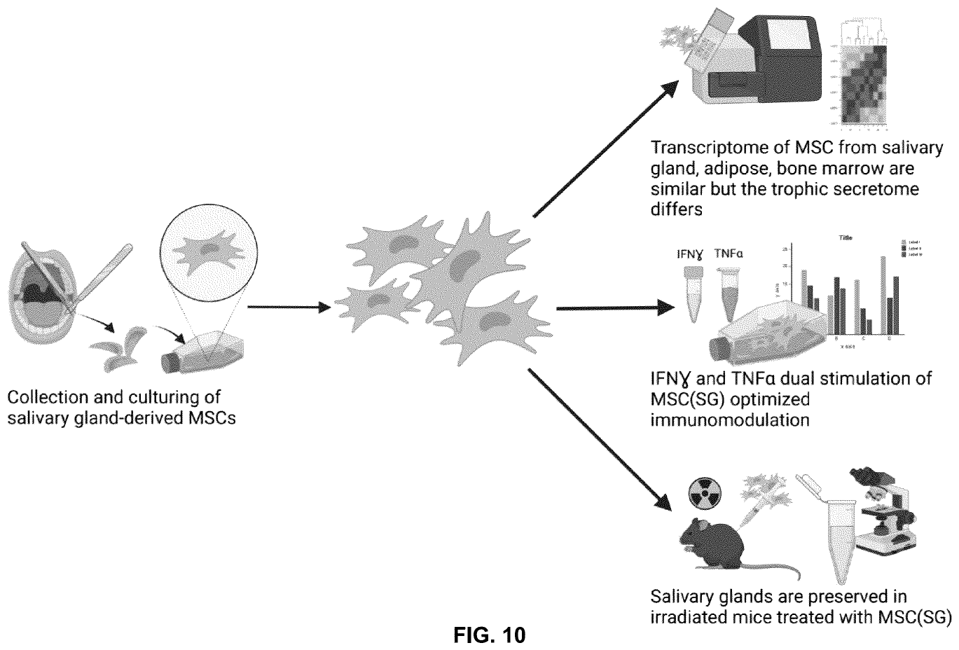

MSCs are a burgeoning therapeutic option for patients suffering from radiation-induced xerostomia. MSC(BM) and MSC(AD) are currently under study as treatment for radiation induced xerostomia (Blitzer et al. “Marrow-Derived Autologous Stromal Cells for the Restoration of Salivary Hypofunction (MARSH): A pilot, first-in-human study of interferon gamma-stimulated marrow mesenchymal stromal cells for treatment of radiation-induced xerostomia,” Cytotherapy. 2023, 25, 1139-1144; Grønhøj et al. “Safety and Efficacy of Mesenchymal Stem Cells for Radiation-Induced Xerostomia: A Randomized, Placebo-Controlled Phase 1/2 Trial (MESRIX),” Int J Radiat Oncol Biol Phys. 2018, 101, 581-592). We report on MSC(SG) as a cell source for treatment of radiation-induced xerostomia. For the first time we compare the phenotype and efficacy of MSC(BM), MSC(AD), and MSC(SG) at baseline and with IFNγ and/or TNFα stimulation. We showed that IFNγ and TNFα pre-licensed MSC(SG) administered intraglandularly protect saliva production and glandular tissue after radiation. This protective effect seemed superior to vehicle pre-licensed MSCs from other tissue. Although the transcriptomic profile was overall similar between MSCs regardless of source, the trophic secretome differed slightly. Dual cytokine pre-licensing with IFNγ and TNFα markedly increased the production of R-spondin 3, a key component of the secretome that allows for intestinal niche progenitor cell repopulation, and markers of MSC immunomodulatory activity. We conclude that MSC(SG), particularly after cytokine pre-licensing, are a feasible and enhanced MSC source for treatment of radiation-induced xerostomia (FIG. 10).

In other organ systems, there is growing recognition that MSCs support local niche progenitor cell replication and differentiation with trophic factors such as Wnts and R-Spondins. R-Spondins amplify canonical Wnt signaling (Yan et al. “The intestinal stem cell markers Bmi1 and Lgr5 identify two functionally distinct populations,” Proc Natl Acad Sci USA. 2012, 109, 466-471; Kim et al. “Mitogenic influence of human R-spondin1 on the intestinal epithelium,” Science. 2005, 309, 1256-1259; Ootani et al. “Sustained in vitro intestinal epithelial culture within a Wnt-dependent stem cell niche,” Nat Med. 2009, 15, 701-706; Schuijers et al. “Ascl2 acts as an R-spondin/Wnt-responsive switch to control stemness in intestinal crypts,” Cell Stem Cell. 2015, 16, 158-170). In the intestines, the progenitor crypt cell is Lgr5+. Study of Wnts and R-Spondins in intestinal epithelium progenitor cells showed that Wnts could not induce Lgr5+ progenitor self-renewal alone but rather Wnts were needed to maintain R-Spondin receptors (Yan et al. “Non-equivalence of Wnt and R-spondin ligands during Lgr5(+) intestinal stem-cell self-renewal” Nature. 2017, 545, 238-242). The presence of R-Spondin receptors drives progenitor cell expansion. R-spondin-3 production from stromal cells is required for injury repair; mice without R-Spondin 3 have no ability to repair after intestinal injury (Harnack et al. “R-spondin 3 promotes stem cell recovery and epithelial regeneration in the colon,” Nat Commun. 2019, 10, 4368). Interestingly, we found that under dual stimulatory conditions, there was an increase in R-Spondin 3 in all MSC groups, but significantly in MSC(SG). This indicates that dual stimulation might optimize key trophic factors to allow recovery of injured local epithelium.

Post-radiation, SGs have increased CD3+ T cells, particularly granzyme B-positive cytotoxic cells (Teymoortash et al. “Lymphocyte subsets in irradiation-induced sialadenitis of the submandibular gland,” Histopathology. 2005, 47, 493-500). In addition to the trophic secretome, we found surrogate marker of MSC immunomodulatory effects, IDO, PD-L1, and ICAM were clearly optimized in the IFNγ and TNFα condition over the IFNγ alone; yet, there was no clear difference between IFNγ alone and combined IFNγ and TNFα regarding ability to suppress T-cell proliferation. This might have represented robust T-cell stimulation with PHA, which might have masked subtle differences by cytokine treatment conditions. Nevertheless, cytokine pre-licensing is likely to optimize MSC immunomodulatory effects.

MSCs have been used to treat radiation in mouse models (Guan et al. “Efficacy of mesenchymal stem cell therapy in rodent models of radiation-induced xerostomia and oral mucositis: a systematic review,” Stem Cell Res Ther. 2023, 14, 82). About half of these studies used intraglandular injections. All used either MSC(BM) or MSC(AD). We describe for the first time the use of MSC(SG). Furthermore, despite the use of pre-licensing in other indications and in human trials, side-by-side comparison of MSC source and cytokine pre-licensing have never been reported. We found that MSC(SG) persist for at least two weeks, which is akin to what has been reported for intraperitoneal or subcutaneous injection persisting at least 10 days (Giri et al. “Mesenchymal stromal cell therapeutic potency is dependent upon viability, route of delivery, and immune match,” Blood Adv. 2020, 4, 1987-1997). In contrast, MSCs given intravenously persist for three days or less (Chinnadurai et al. “Cryopreserved Mesenchymal Stromal Cells Are Susceptible to T-Cell Mediated Apoptosis Which Is Partly Rescued by IFNγ Licensing,” Stem Cells. 2016, 34, 2429-2442). We found that pre-licensing MSC(SG) with either IFNγ or both IFNγ and TNFα prevented SG dysfunction beyond MSC(SG) treated with vehicle. Given the advantage of dual IFNγ/TNFα therapy on the immunomodulatory and trophic profile of MSC(SG), we proceeded with testing combination therapy among MSCs by source. Interestingly, when we compare dual therapy by MSC source, MSC(SG) showed the greatest salivary flow preservation. Further, we showed that MSCs treated with IFNγ and TNFα preserve SG tissue of mice.

We found that the transcriptional phenotype of MSCs, regardless of the source, are quite similar, with only a few key differences. MSC(SG) has more G-protein coupled receptors related to chemokine signaling, prostaglandin and endothelin, indicating MSC(SG) may be more sensitive to local inflammation. MSC(SG) have increased transcripts related to interferon signaling compared to MSC(BM), even when we focused only on MSC(SG) from healthy participants. Past studies of palatine tonsil MSCs, when compared to MSC(BM) and MSC(AD), showed similar results (Cho et al. “RNA sequencing reveals a transcriptomic portrait of human mesenchymal stem cells from bone marrow, adipose tissue, and palatine tonsils,” Sci Rep. 2017, 7, 17114). This finding might reflect the fact that salivary glands interface with the environment and are a common site of persistent viral infection. Compared to MSC(BM), MSC(SG) has fewer transcripts related to IRE-1 alpha chaperone, which is related to the Unfolded Protein Response. MSC aging is promoted by oxidative stress and abnormal Unfolded Protein Response. Further, chronic Unfolded Protein Response stimulation (indicating the unfolded protein response could not be resolved) causes apoptosis. Thus, differential Unfolded Protein Response between MSCs might reflect differential aging and oxidative stress by MSC source. Finally, MSC(AD) and MSC(SG) had more neuronal related transcripts compared to MSC(BM), such as Slitrks, that are responsible for neuron development and growth. This is particularly salient to salivary gland tissue, because salivation is reliant on sympathetic and parasympathetic innervation.

This Example compares MSC(SG) to other standard MSC sources used to treat radiation induced xerostomia in mouse models. Furthermore, we report on the effects of cytokine stimulation across these cell types on phenotype and function of the MSCs in the context of radiation induced xerostomia. We cannot confirm the baseline demographics of our MSC(BM) and MSC(AD) derived from humans; however, we saw no major differences of any one cell source in each group, indicating there were likely no major aberrant subjects included. Our transcriptomic approach provided an unbiased view of potential mechanistic differences by MSC source; however, given the focus on therapeutic intervention no further mechanistic studies were performed. Further studies can be performed to understand the mechanism of how our MSC(SG) alleviate SG dryness and preserve SG tissue in mice.

This Example provides evidence for the use of dual pre-licensed MSC(SG) for the treatment or prevention of radiation induced xerostomia. Radiation induced xerostomia patients are already being treated with MSC(BM) pre-licensed with IFNγ. The current study supports future clinical trials using dual pre-licensed MSC(SG) based on the ease and cost of procurement, secretome, and immunomodulatory capabilities of these cells.

What is claimed is:

1. A method of treating xerostomia in a subject, the method comprising administering to the subject a therapeutically effective amount of a composition comprising mesenchymal stromal cells (MSCs), wherein the MSCs are treated with a combination of interferon gamma (IFNγ) and tumor necrosis factor alpha (TNFα) prior to administration.

2. The method of claim 1, wherein the MSCs have an increased secretion of R-spondin 3 after being treated with the combination of IFNγ and TNFα.

3. The method of claim 1, wherein the MSCs are cryopreserved after being treated with the combination of IFNγ and TNFα, and are cryo-recovered prior to administration.

4. The method of claim 3, wherein the MSCs after being cryo-recovered have an increased expression of one or more immunomodulatory factors as compared to MSCs not being treated with the combination of IFNγ and TNFα.

5. The method of claim 1, wherein the MSCs are isolated from a tissue prior to being treated by the combination of IFNγ and TNFα, wherein the tissue is selected from the group consisting of salivary gland, bone marrow, umbilical cord, and an adipose tissue.

6. The method of claim 5, wherein the tissue is salivary gland.

7. The method of claim 1, wherein the MSCs are allogeneic to the subject.

8. The method of claim 1, wherein the MSCs are autologous to the subject.

9. The method of claim 1, wherein the MSCs are syngeneic to the subject.

10. The method of claim 1, wherein the IFNγ and TNFα are human IFNγ and TNFα.

11. The method of claim 1, wherein the IFNγ and TNFα are recombinant IFNγ and TNFα.

12. The method of claim 1, wherein the xerostomia is associated with Sjögren's syndrome.

13. The method of claim 1, wherein the xerostomia is associated with graft-versus-host disease.

14. The method of claim 1, wherein the xerostomia is age-related xerostomia.

15. The method of claim 1, wherein the xerostomia is radiation-induced xerostomia.

16. The method of claim 1, wherein the xerostomia is medication-induced xerostomia, wherein the medication comprises one or more of antidepressant, anticholinergic, antihypertensive, antihistamine, and chemotherapy drugs.

17. The method of claim 1, wherein the subject is a mammal, including a human.
